(19) United States
(12) Patent Application Publication
(10) Pub. No.: US 2014/0170020 A1
(43) Pub. Date: Jun. 19, 2014

(54) STERILIZATION APPARATUS, STERILIZATION METHOD

(71) Applicants: CANON MARKETING JAPAN KABUSHIKI KAISHA, Tokyo (JP); KABUSHIKI KAISHA ELQUEST, Chiba (JP); CANON LIFECARE SOLUTIONS KABUSHIKI KAISHA, Osaka (JP)

(72) Inventor: Atsushi Hiruta, Chiba (JP)

(73) Assignees: CANON MARKETING JAPAN KABUSHIKI KAISHA, Tokyo (JP); KABUSHIKI KAISHA ELQUEST, Chiba (JP); CANON LIFECARE SOLUTIONS KABUSHIKI KAISHA, Osaka (JP)

(21) Appl. No.: 14/105,058

(22) Filed: Dec. 12, 2013

(30) Foreign Application Priority Data
Dec. 13, 2012 (JP) 2012-272252
May 20, 2013 (JP) 2013-106660

(51) Int. Cl. A61L 2/26; A61L 2/16

(52) U.S. Cl. CPC A61L 2/26; A61L 2/16
USPC 422/28; 422/292

(57) ABSTRACT

There is provided a sterilization apparatus capable of delaying degradation of sterilant in a cartridge or a tank in which an extraction tube is inserted. A sterilization apparatus extracting sterilant from a cartridge containing the sterilant and sterilizing a sterilization target includes an extraction tube configured to extract the sterilant from the cartridge, and a movement unit configured to move the extraction tube so that a position of the extraction tube with respect to the cartridge in a case of extracting the sterilant in the cartridge by the extraction tube and a position of the extraction tube with respect to the cartridge in a case of waiting for extraction of the sterilant from the cartridge by the extraction tube to perform following sterilization processing with the same cartridge may differ from each other.

### BACKGROUND

1. Field

Aspects of the present invention generally relate to a sterilization apparatus and a sterilization method, and more specifically relates to a technique for delaying degradation of sterilant in a cartridge or a tank.

2. Description of the Related Art

Medical devices such as syringes and surgical tools cannot be reused without sterilization after use since pathogens may attach to the medical devices and may have an effect on a human body. For this reason, there exist sterilization apparatuses that sterilize targets requiring sterilization such as medical devices.

As one of such sterilization apparatuses, Japanese Unexamined Patent Application Publication (Translation of PCT Application) No. 08-505787 discusses a sterilization apparatus and a sterilization method in which a target is sterilized with use of hydrogen peroxide as sterilant.

However, sterilant (e.g., sterilant containing a hydrogen peroxide solution), in a cartridge to be used in a sterilization apparatus performing sterilization processing to a sterilization target by suctioning as much sterilant as a dose for a sterilization processing operation with use of an extraction tube extracting the sterilant from the cartridge in which as much sterilant as doses for several sterilization processing operations is contained in a bottle, is gradually degraded as time goes by.

Accordingly, in a case where the sterilization processing is performed with use of the sterilant in the cartridge after the elapse of a certain period of time after production, a sufficient sterilization effect may not be achieved.

Also, when the extraction tube is inserted to open the cartridge for the sterilization processing, substances in the atmosphere, such as dust, may enter the cartridge, and degradation of the sterilant in the cartridge may further be accelerated.

Further, when the extraction tube, which is made of a metal such as stainless steel to prevent corrosion by the sterilant, is inserted into the cartridge for the sterilization processing, degradation of the sterilant may further be accelerated by an interaction between components contained in the inserted extraction tube and the sterilant.

To address this issue, it is conceivable to pull out the extraction tube of the cartridge still containing the sterilant in which the extraction tube has been inserted. However, when the extraction tube is pulled out of the cartridge, a hole is produced at a part of the cartridge in which the extraction tube has been inserted.

Accordingly, substances in the atmosphere may enter the cartridge through the hole produced in the cartridge, and degradation of the sterilant in the cartridge may be accelerated.

Also, part of the sterilant in the cartridge may be gasified from the hole produced in the cartridge and flow in the sterilization apparatus. In a case where the gasified sterilant flows in the sterilization apparatus, the gasified sterilant and metals of respective parts in the sterilization apparatus may react with one another, for example, to accelerate deterioration of the respective parts.

In this manner, while the extraction tube and the sterilant in the cartridge contact each other by insertion of the extraction tube in the cartridge containing the sterilant for extraction of the sterilant, degradation of the sterilant in the cartridge is accelerated.

Accordingly, the longer a period of the contact between the extraction tube and the sterilant by insertion of the extraction tube in the cartridge is, the shorter a period in which the sterilization processing that can exert a sufficient sterilization effect can be performed with use of the sterilant will be.

Under such circumstances, in a mechanism for inserting the extraction tube in the cartridge in which as much sterilant as doses for several sterilization processing operations is filled in a bottle, extracting as much sterilant as a dose from the cartridge, and performing the sterilization processing with use of the extracted sterilant, a mechanism for delaying degradation of the sterilant in the cartridge in which the extraction tube is inserted to prevent easy shortening of the period in which the sterilization processing that can exert a sufficient sterilization effect can be performed is required.

Similarly, in a mechanism for inserting the extraction tube in a tank, not the cartridge, in which the sterilant suctioned from the cartridge is accumulated, extracting as much sterilant as a dose from the tank, and performing the sterilization processing with use of the extracted sterilant, a mechanism for delaying degradation of the sterilant in the tank in which the extraction tube is inserted to prevent easy shortening of the period in which the sterilization processing that can exert a sufficient sterilization effect can be performed is required from a similar reason to that in the case of the cartridge.

### SUMMARY

Aspects of the present invention are generally directed to a sterilization apparatus and a sterilization method capable of delaying degradation of sterilant in a cartridge or a tank in which an extraction tube is inserted.

According to an aspect of the present invention, a sterilization apparatus extracting sterilant from a cartridge containing the sterilant and sterilizing an target includes an extraction tube configured to extract the sterilant from the cartridge, and a movement unit configured to move the extraction tube so that a position of the extraction tube against the cartridge in a case of extracting the sterilant in the cartridge by the extraction tube and a position of the extraction tube against the cartridge in a case of waiting for extraction of the sterilant from the cartridge by the extraction tube to perform following sterilization processing with the same cartridge differ from each other.

Further features and aspects of the present disclosure will become apparent from the following detailed description of exemplary embodiments with reference to the attached drawings.

### BRIEF DESCRIPTION OF THE DRAWINGS

FIG. 1 is a front view of an outer appearance of a sterilization apparatus according to a first exemplary embodiment.

FIG. 2 is a block diagram illustrating an example of a hardware configuration of the sterilization apparatus according to the first exemplary embodiment.

FIG. 3 illustrates an example of a screen to be displayed on a display unit 102 of a sterilization apparatus 100.

FIG. 4 (composed of FIGS. 4A and 4B) is a flowchart illustrating an example of each step in sterilization processing by the sterilization apparatus according to the first exemplary embodiment.

FIG. 5 is a flowchart illustrating an example of detailed processing for the sterilization processing illustrated in step S111 in FIG. 4.

FIG. 6 is a flowchart illustrating an example of detailed processing in a pre-sterilization step illustrated in step S501 in FIG. 5.

FIG. 7 (composed of FIGS. 7A and 7B) is a flowchart illustrating an example of detailed processing in a sterilization step illustrated in step S502 in FIG. 5.

FIG. 8 is a flowchart illustrating an example of detailed processing in a ventilation step illustrated in step S503 in FIG. 5.

FIG. 9 is a flowchart illustrating an example of detailed processing for sterilant evacuation processing in step S114 in FIG. 4.

FIG. 10 is an example of a block configuration diagram illustrating a hardware configuration of a concentration furnace 208, a valve (V1) 211, a valve (V3) 212, a valve (V4) 213, a measuring tube 214, a valve (V2) 215, a gasification furnace 216, a valve (V5) 217, and a valve (V9) 227 in the sterilization apparatus 100 according to the first exemplary embodiment.

FIG. 11 illustrates an example of a cartridge attachment request screen 1101 to be displayed on the display unit 102 of the sterilization apparatus 100.

FIG. 12 is a side view of a cartridge 205 for the sterilant to be used in the sterilization apparatus according to the first exemplary embodiment.

FIG. 13 is a cross-sectional view along a cross-section 1 of the cartridge 205 when a tip of an extraction needle 203-A has been inserted to a bottom of the cartridge 205 to suction the sterilant in the cartridge 205.

FIG. 14 is a block diagram illustrating an example of a hardware configuration of the sterilization apparatus according to a second exemplary embodiment.

FIG. 15 is a cross-sectional view along the cross-section 1 illustrating an example of the cartridge 205 whose unsealing portion is sealed by the extraction needle 203-A that has been moved so that the tip of the extraction needle 203-A may be located at a position in the cartridge 205 in which the extraction needle 203-A does not dip in the sterilant.

FIG. 16 is a block diagram illustrating an example of a hardware configuration of the sterilization apparatus according to a third exemplary embodiment.

FIG. 17 is a block diagram illustrating an example of a hardware configuration of the sterilization apparatus according to a fourth exemplary embodiment.

### DESCRIPTION OF THE EMBODIMENTS

A sterilization apparatus, a sterilization method according to exemplary embodiments will be described with reference to the drawings.

Hereinafter, a first exemplary embodiment of the sterilization apparatus will be described with reference to the drawings.

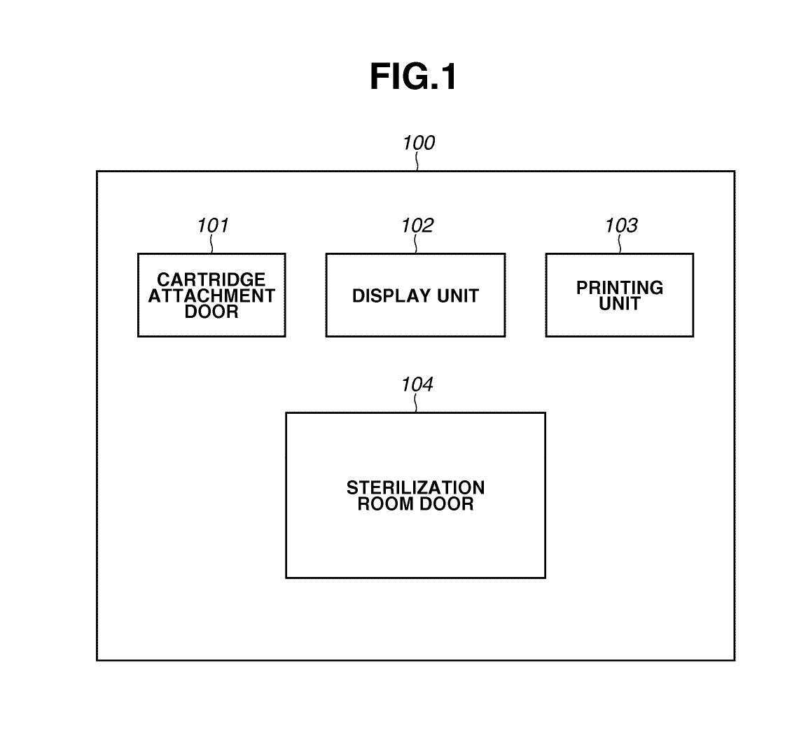

First, an outer appearance of the sterilization apparatus according to the present exemplary embodiment will be described with reference to FIG. 1.

FIG. 1 is a front view of the outer appearance of the sterilization apparatus according to the present exemplary embodiment. A sterilization apparatus 100 according to the present exemplary embodiment includes a cartridge attachment door 101, a display unit 102, a printing unit 103, and a sterilization room door 104.

The cartridge attachment door 101 is a door for attaching a cartridge, which is a container filled with sterilant (e.g., a solution containing hydrogen peroxide). When a user opens the cartridge attachment door 101, there exists a place for attachment of the cartridge, to which the user can attach the cartridge.

The display unit 102 is a touch panel display screen such as a liquid crystal display.

The printing unit 103 is a printer for printing a history of sterilization processing and a sterilization result on a printing paper and prints the history of the sterilization processing and the sterilization result on the printing paper as needed.

The sterilization room door 104 is a door through which the user puts a target to be sterilized (sterilization target) such as a medical device in a sterilization room for sterilizing the sterilization target. When the user opens the sterilization room door 104, there exists the sterilization room. The user can put the sterilization target in the sterilization room by putting the sterilization target in the sterilization room and closing the sterilization room door 104.

The sterilization room is a casing with a predetermined capacity. As for the air pressure (pressure) in the sterilization room, the pressure ranging from the atmospheric pressure to the vacuum can be kept. In addition, as for a temperature in the sterilization room, a predetermined temperature range is kept during the sterilization processing.

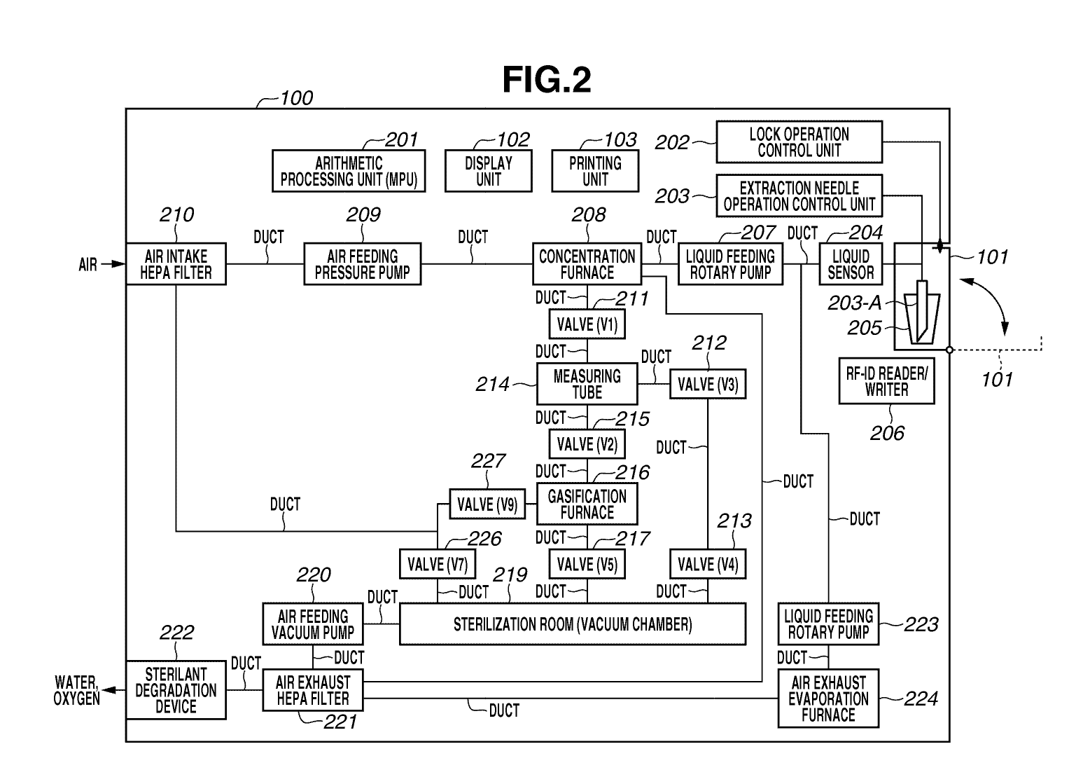

Next, an example of a hardware configuration of the sterilization apparatus according to the present exemplary embodiment will be described with reference to FIG. 2.

FIG. 2 is a block diagram illustrating an example of a hardware configuration of the sterilization apparatus according to the present exemplary embodiment.

The sterilization apparatus 100 according to the present exemplary embodiment includes an arithmetic processing unit (such as a micro-processing unit (MPU)) 201, the display unit 102, the printing unit 103, a lock operation control unit 202, an extraction needle 203-A, an extraction needle operation control unit 203, the cartridge attachment door 101, a liquid sensor 204, a cartridge 205, a radio frequency identification (RF-ID) reader/writer 206, a liquid feeding rotary pump 207, a concentration furnace 208, an air feeding pressure pump 209, an air intake high efficiency particulate air (HEPA) filter 210, a valve (V1) 211, a valve (V3) 212, a valve (V4) 213, a measuring tube 214, a valve (V2) 215, a gasification furnace 216, a valve (V5) 217, a valve (V9) 227, a valve (V7) 226, a sterilization room (also referred to as a vacuum chamber) 219, an air feeding vacuum pump 220, an air exhaust HEPA filter 221, sterilant degradation device 222, a liquid feeding rotary pump 223, and an air exhaust evaporation furnace 224.

The sterilization apparatus 100 is an apparatus that sterilizes a target by taking out the sterilant from the cartridge 205 containing the sterilant.

The arithmetic processing unit (such as the MPU) 201 performs arithmetic processing to control respective hardware units constituting the sterilization apparatus 100 as described below.

The arithmetic processing unit (such as the MPU) 201 is an example of application of a control unit according to the present exemplary embodiment.

Since the display unit 102, the printing unit 103, and the cartridge attachment door 101 have been described with reference to FIG. 1, description thereof is omitted here.

The lock operation control unit 202 is a unit that performs locking and unlocking operations of the cartridge attachment door 101. The lock operation control unit 202 prevents the cartridge attachment door 101 from being opened by locking the cartridge attachment door 101 and allows the cartridge attachment door 101 to be opened by unlocking the cartridge attachment door 101.

The cartridge 205 is a sealed container in which the sterilant (hydrogen peroxide or a hydrogen peroxide solution liquid) is filled. Also, the cartridge 205 is provided on a lower side thereof with an RF-ID storage medium, and the storage medium has stored therein a serial number as information for identification of the cartridge 205, a manufacturing date of the cartridge 205, date and time when the cartridge 205 is first used in the sterilization apparatus 100 (first use date and time), and a remaining amount of the sterilant filled in the cartridge 205.

This RF-ID is a storage medium having stored therein data regarding disposal of the sterilant in the cartridge 205 (all or any of data including the serial number, the manufacturing date, the first use date and time, and the remaining amount of the sterilant).

The extraction needle operation control unit 203 is a unit that controls (drives) the extraction needle 203-A (injection needle) for suction of the sterilant in the cartridge 205 to move the extraction needle 203-A for being inserting in an upper portion of the cartridge 205.

This extraction needle operation control unit 203 is an example of application of a movement unit according to the present exemplary embodiment.

More specifically, the extraction needle operation control unit 203 (movement unit) moves the extraction needle 203-A (extraction tube) so that a position of the extraction tube against the cartridge (205) in a case of extracting the sterilant in the cartridge by the extraction tube and a position of the extraction tube against the cartridge in a case of waiting for extraction of the sterilant from the cartridge by the extraction tube to perform the following sterilization processing with the same cartridge may differ from each other.

More specifically, in a case where the extraction needle operation control unit 203 inserts the extraction needle 203-A (injection needle) for suction of the sterilant in the cartridge 205 in the upper portion of the cartridge 205, the extraction needle operation control unit 203 can insert the extraction needle 203-A (injection needle) in the upper portion of the cartridge 205 by controlling the extraction needle 203-A (injection needle) to lower the extraction needle 203-A (injection needle) toward the cartridge 205 from the upper portion of the cartridge 205. Also, in a case where the extraction needle operation control unit 203 pulls the extraction needle 203-A (injection needle) out of the cartridge 205, the extraction needle operation control unit 203 can pull the extraction needle 203-A (injection needle) out of the cartridge 205 by controlling the extraction needle 203-A (injection needle) to raise the extraction needle 203-A (injection needle) to the upper portion of the cartridge 205.

The extraction needle 203-A is a straw (thin tube) for suctioning and extracting the sterilant in the cartridge 205. The extraction needle 203-A is an example of application of an extraction tube according the present exemplary embodiment extracting the sterilant from the cartridge 205.

This extraction needle 203-A is made of a metal such as stainless steel to prevent corrosion by the sterilant, which is a solution containing hydrogen peroxide, for example.

The liquid sensor 204 is a device that detects whether the liquid sterilant in the cartridge 205 passes through tubes (ducts) communicating (connecting) with the liquid feeding rotary pump 207 and liquid feeding rotary pump 223 via the extraction needle 203-A (injection needle). Specifically, the liquid sensor 204 can detect from spectra obtained by irradiation of the tubes with infrared rays whether the sterilant passes through the tubes.

The RF-ID reader/writer 206 is a device that can read the serial number, the manufacturing date, the first use date and time, and the remaining amount of the sterilant from the RF-ID provided on the lower side of the cartridge 205. The RF-ID reader/writer 206 is also a device that can write the first use date and time and the remaining amount of the sterilant in the RF-ID provided on the lower side of the cartridge 205. Also, the RF-ID reader/writer 206 is disposed at a lower portion of a cartridge attaching position behind the cartridge attachment door 101, and can read the data in the RF-ID provided on the lower side of the cartridge 205, and can write the data such as the first use date and time and the remaining amount of the sterilant in the RF-ID.

The liquid feeding rotary pump 207 communicates (connects) with the concentration furnace 208 by a duct and communicates with the liquid sensor 204 by a duct. The liquid feeding rotary pump 207 is a device that suctions the liquid sterilant in the cartridge 205 by a pump and feeds the sterilant to the concentration furnace 208 via the duct. In addition, the liquid feeding rotary pump 207 can suction a predetermined amount of the sterilant from the cartridge 205 in cooperation with the liquid sensor 204.

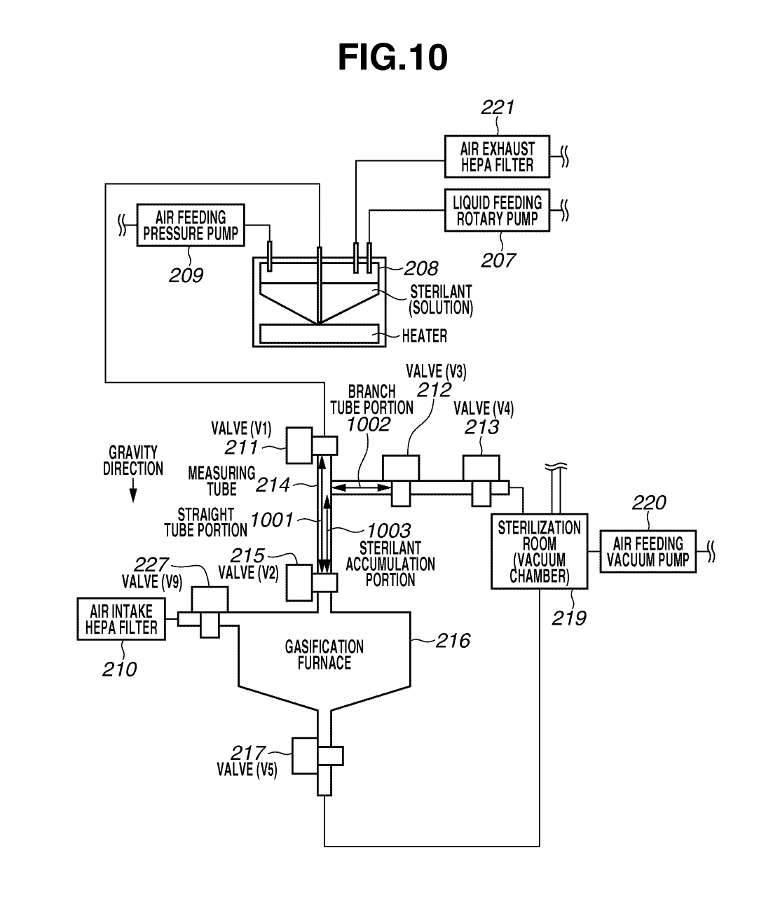

The concentration furnace 208 communicates with the liquid feeding rotary pump 207, the air feeding pressure pump 209, the measuring tube 214, and the air exhaust HEPA filter 221 by ducts, respectively. As described below with reference to FIG. 10, the concentration furnace 208 heats by a heater the sterilant fed from the liquid feeding rotary pump 207 via the duct and evaporates (gasifies) water contained in the sterilant to concentrate the sterilant. In addition, the gasified water is pushed into the duct communicating with the air exhaust HEPA filter 221 by air fed from the air feeding pressure pump 209 via the duct, and is exhausted from the concentration furnace 208. Also, the valve (V1) 211 is provided in the middle of the duct between the measuring tube 214 and the concentration furnace 208.

The air feeding pressure pump 209 communicates with the concentration furnace 208 and the air intake HEPA filter 210 by ducts, respectively. The air feeding pressure pump 209 is a device that lets external air (air) of the sterilization apparatus 100 pass through the duct communicating with the air intake HEPA filter 210 via the air intake HEPA filter 210 to feed the air to the concentration furnace 208.

The air intake HEPA filter 210 communicates with the air feeding pressure pump 209, the sterilization room. 219, and the gasification furnace 216 by ducts, respectively. The air intake HEPA filter 210 filters and cleans the external air (air) outside the sterilization apparatus 100 by a HEPA filter to remove dust, dirt, and bacteria in the air. The cleaned air is fed to the concentration furnace 208 via the duct by the air feeding pressure pump 209. The cleaned air is let to pass through the duct communicating with the gasification furnace 216 to be fed to the gasification furnace 216 and is let to pass through the duct communicating with the sterilization room. 219 to be fed to the sterilization room 219. In other words, the air intake HEPA filter 210 communicates with the external air (air) outside the sterilization apparatus 100. Thus, the duct between the air feeding pressure pump 209 and the air intake HEPA filter 210, the duct between the sterilization room 219 and the air intake HEPA filter 210, and the duct between the gasification furnace 216 and the air intake HEPA filter 210 communicate with the external air (air) via the air intake HEPA filter 210.

Also, the duct between the air intake HEPA filter 210 and the gasification furnace 216 is provided with the valve (V9) 227. In addition, the duct between the air intake HEPA filter 210 and the sterilization room 219 is provided with the valve (V7) 226.

The valve (V1) 211 is a valve provided in the duct between the concentration furnace 208 and the measuring tube 214, and is a valve enabling communication between the concentration furnace 208 and the measuring tube 214 by the duct when the valve (V1) 211 is opened and disabling communication between the concentration furnace 208 and the measuring tube 214 by the duct when the valve (V1) 211 is closed.

The valve (V3) 212 is a valve provided in the duct between the measuring tube 214 and the sterilization room. 219, and is a valve enabling communication between the measuring tube 214 and the sterilization room 219 by the duct when the valve (V3) 212 is opened and disabling communication between the measuring tube 214 and the sterilization room 219 by the duct when the valve (V3) 212 is closed. Also, this valve (V3) 212 is provided close to the measuring tube 214, and is provided at least at a position further to a side of the measuring tube 214 than the after-mentioned valve (V4) 213.

The valve (V4) 213 is a valve provided in the duct between the measuring tube 214 and the sterilization room. 219, and is a valve enabling communication between the measuring tube 214 and the sterilization room 219 by the duct when the valve (V4) 213 is opened, and disabling communication between the measuring tube 214 and the sterilization room 219 by the duct when the valve (V4) 213 is closed. Also, this valve (V4) 213 is provided close to the sterilization room 219, and is provided at least at a position closer to a side of the sterilization room 219 than the valve (V3) 212.

Although communication of the duct between the measuring tube 214 and the sterilization room 219 is enabled or disabled by opening/closing of the valve (V3) 212 and the valve (V4) 213 in the present exemplary embodiment, communication of the duct between the measuring tube 214 and the sterilization room 219 may be enabled or disabled by opening/closing of either the valve (V3) 212 or the valve (V4) 213.

In other words, either the valve (V3) 212 or the valve (V4) 213 may be provided, and communication of the duct between the measuring tube 214 and the sterilization room 219 may be enabled or disabled by opening/closing of the provided valve.

The measuring tube 214 communicates with the concentration furnace 208, the gasification furnace 216, and the sterilization room 219 by respective in-between ducts.

The measuring tube 214 is a device in which the sterilant flows from the concentration furnace 208 by opening the valve (V1) 211 and that eliminates by the measuring tube 214 unnecessary air suctioned from the cartridge 205 by opening the valve (V3) 212 and the valve (V4) 213 and/or unnecessary air flowing from the air intake HEPA filter 210 in the concentration furnace 208 and flowing from the concentration furnace 208 in the measuring tube 214 by opening the valve (V3) 212 and the valve (V4) 213. Detailed description of the measuring tube 214 will be given below with reference to FIG. 10.

The valve (V2) 215 is a valve provided in the duct between the measuring tube 214 and the gasification furnace 216 and is a valve enabling communication between the measuring tube 214 and the gasification furnace 216 by the duct when the valve (V2) 215 is opened and disabling communication between the measuring tube 214 and the gasification furnace 216 by the duct when the valve (V2) 215 is closed.

The gasification furnace 216 communicates with the measuring tube 214, the air intake HEPA filter 210, and the sterilization room 219 by respective in-between ducts. The gasification furnace 216 is an example of application of a gasification room according to the present exemplary embodiment.

The gasification furnace 216 is a device that gasifies the sterilant by being decompressed by the air feeding vacuum pump 220.

The valve (V5) 217 is a valve provided in the duct between the gasification furnace 216 and the sterilization room 219, and is a valve enabling communication between the gasification furnace 216 and the sterilization room 219 by the duct when the valve (V5) 217 is opened, and disabling communication between the gasification furnace 216 and the sterilization room 219 by the duct when the valve (V5) 217 is closed.

The valve (V9) 227 is a valve provided in the duct between the gasification furnace 216 and the air intake HEPA filter 210, and is a valve enabling communication between the gasification furnace 216 and the air intake HEPA filter 210 by the duct when the valve (V9) 227 is opened, and disabling communication between the gasification furnace 216 and the air intake HEPA filter 210 by the duct when the valve (V9) 227 is closed. In other words, the valve (V9) 227 is a valve that can open/close communication between the gasification furnace 216 and the external air (atmospheric air).

The valve (V7) 226 is a valve provided in the duct between the sterilization room 219 and the air intake HEPA filter 210, and is a valve enabling communication between the sterilization room 219 and the air intake HEPA filter 210 by the duct when the valve (V7) 226 is opened, and disabling communication between the sterilization room 219 and the air intake HEPA filter 210 by the duct when the valve (V7) 226 is closed. In other words, the valve (V7) 226 is a valve that can open/close communication between the sterilization room 219 and the external air (atmospheric air).

The sterilization room (also referred to as a vacuum chamber) 219 is a casing with a predetermined capacity that sterilizes a sterilizing sterilization target such as a medical device as has been described in FIG. 1. As for the pressure in the sterilization room 219, the pressure ranging from the atmospheric pressure to the vacuum can be kept. Further, as for a temperature in the sterilization room 219, a predetermined temperature range is kept during the sterilization processing. Further, the sterilization room 219 is provided therein with a pressure sensor to measure pressure (air pressure) in the sterilization room 219. The sterilization apparatus 100 determines whether the pressure (air pressure) in the sterilization room 219 is a predetermined air pressure by using the air pressure in the sterilization room 219 measured by this pressure sensor.

The air feeding vacuum pump 220 is a device that suctions gas in spaces in the sterilization room 219, in the gasification furnace 216, in the measuring tube 214, in the duct between the measuring tube 214 and the gasification furnace 216, in the duct between the gasification furnace 216 and the sterilization room 219, and in the duct between the measuring tube 214 and the sterilization room 219 to decompress the respective spaces into vacuum states (states in the spaces filled with gas with lower pressure than the atmospheric pressure).

The air feeding vacuum pump 220 communicates with the sterilization room 219 by a duct, and communicates with the air exhaust HEPA filter 221 by a duct.

The air exhaust HEPA filter 221 communicates with the air feeding vacuum pump 220 by a duct. The air exhaust HEPA filter 221 also communicates with the air exhaust evaporation furnace 224 by a duct. The air exhaust HEPA filter 221 also communicates with the sterilant degradation device 222 by a duct. The air exhaust HEPA filter 221 also communicates with the concentration furnace 208 by a duct.

The air exhaust HEPA filter 221 filters and cleans gas suctioned from the sterilization room 219 by the air feeding vacuum pump 220 by a HEPA filter to remove dust, dirt, and bacteria in the gas fed from the duct communicating with the air feeding vacuum pump 220. The cleaned gas passes through the duct between the sterilant degradation device 222 and the air exhaust HEPA filter 221, and is fed to the sterilant degradation device 222, and the sterilant degradation device 222 degrades molecules of the sterilant contained in the gas, and discharges the degraded molecules out of the sterilization apparatus 100.

The air exhaust HEPA filter 221 also cleans gas to be exhausted from the concentration furnace 208 by the duct between the concentration furnace 208 and the air exhaust HEPA filter 221. This gas is gasified water by heating of the sterilant in the concentration furnace 208. Since the air contains a small amount of the sterilant, the air passes through the duct between the sterilant degradation device 222 and the air exhaust HEPA filter 221, and is fed to the sterilant degradation device 222. Subsequently, the sterilant degradation device 222 degrades molecules of the sterilant contained in the gas, and discharges the degraded molecules out of the sterilization apparatus 100.

The sterilant degradation device 222 is an example of application of a disposal unit according to the present exemplary embodiment disposing the sterilant contained in the cartridge 205 attached to the sterilization apparatus 100.

The air exhaust HEPA filter 221 also cleans the gasified sterilant to be fed from the air exhaust evaporation furnace 224 through the duct between the air exhaust evaporation furnace 224 and the air exhaust HEPA filter 221. The cleaned sterilant (gas) then passes through the duct between the sterilant degradation device 222 and the air exhaust HEPA filter 221, and is fed to the sterilant degradation device 222. Subsequently, the sterilant degradation device 222 degrades molecules of the sterilant contained in the gas, and discharges the degraded molecules out of the sterilization apparatus 100.

The air exhaust HEPA filter 221 cleans gas fed through the duct to enable preventing easy accumulation of dirt and foreign matters in the sterilant degradation device 222 and extending a product lifetime of the sterilant degradation device 222.

The sterilant degradation device 222 communicates with the air exhaust HEPA filter 221 by an in-between duct. The sterilant degradation device 222 degrades molecules of the sterilant contained in gas fed from the duct between the sterilant degradation device 222 and the air exhaust HEPA filter 221, and discharges the molecules generated by degradation out of the sterilization apparatus 100.

The sterilant degradation device 222 is a device that degrades the sterilant. For example, in a case where the sterilant is hydrogen peroxide or a hydrogen peroxide solution, the sterilant degradation device 222 can degrade gasified hydrogen peroxide into water and oxygen by using manganese dioxide as a catalyst.

The liquid feeding rotary pump 223 communicates with the air exhaust evaporation furnace 224 by a duct, and communicates with the liquid sensor 204 by a duct.

The liquid feeding rotary pump 223 is a device that suctions all the liquid sterilant in the cartridge 205 by a pump, and feeds all the sterilant fed through the duct between the liquid sensor 204 and the liquid feeding rotary pump 223 to the air exhaust evaporation furnace 224 through the duct between the liquid feeding rotary pump 223 and the air exhaust evaporation furnace 224.

The air exhaust evaporation furnace 224 communicates with the liquid feeding rotary pump 223 by a duct, and communicates with the air exhaust HEPA filter 221 by a duct.

The air exhaust evaporation furnace 224 heats by a heater provided in the air exhaust evaporation furnace 224 all the liquid sterilant in the cartridge 205 to be fed through the duct between the liquid feeding rotary pump 223 and the air exhaust evaporation furnace 224 and gasifies all the sterilant. Subsequently, the gasified sterilant is fed to the air exhaust HEPA filter 221 through the duct between the air exhaust HEPA filter 221 and the air exhaust evaporation furnace 224.

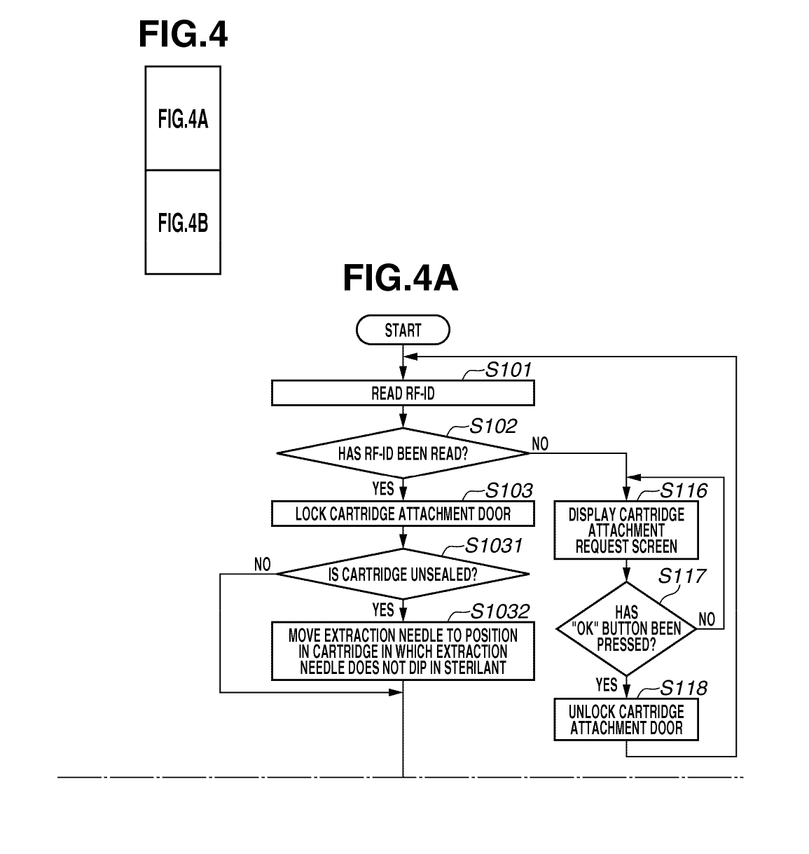

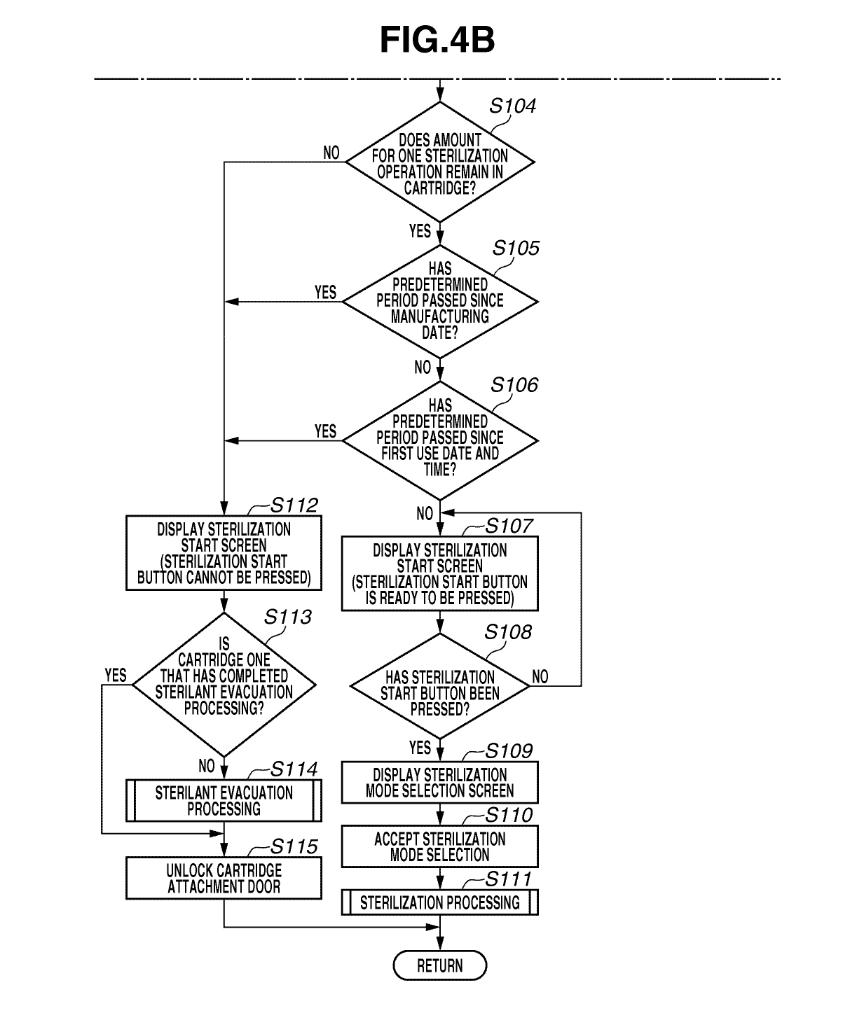

Next, an example of each step in the sterilization processing by the sterilization apparatus 100 according to the present exemplary embodiment will be described with reference to FIG. 4 (composed of FIGS. 4A and 4B).

Each step (process) illustrated in FIG. 4 is performed by control of operations of each device in the sterilization apparatus 100 by the arithmetic processing unit 201 of the sterilization apparatus 100.

That is, the arithmetic processing unit 201 of the sterilization apparatus 100 executes a program readable and executable by the arithmetic processing unit 201 of the sterilization apparatus 100 to control operations of each device and execute each step (process) illustrated in the figure.

FIG. 4 is a flowchart illustrating an example of each step in the sterilization processing by the sterilization apparatus 100 according to the present exemplary embodiment.

In step S101, when the sterilization apparatus 100 is powered on, the RF-ID reader/writer 206 (reading unit/writing unit) first reads data from the RF-ID (storage medium) provided on the lower side of the cartridge 205.

The RF-ID reader/writer 206 is an example of application of a detection unit according to the present exemplary embodiment for detecting that the cartridge 205 has been attached to the sterilization apparatus 100.

In step S101, the data to be read from the RF-ID (storage medium) includes the serial number as information for identification of the cartridge 205, the manufacturing date of the cartridge 205, the date and time when the cartridge 205 is first used in the sterilization apparatus 100 (first use date and time), and the remaining amount of the sterilant filled in the cartridge 205. That is, the RF-ID (storage medium) provided in the cartridge 205 stored in advance therein the serial number, the manufacturing date, the first use date and time (first use date and time information), and the remaining amount of the sterilant. Meanwhile, the RF-ID of the cartridge 205 to be first used in the sterilization apparatus 100 does not have stored therein the first use date and time (date and time when the cartridge 205 is first used in the sterilization apparatus 100). Thus, the RF-ID of the cartridge 205 to be first used has stored therein the serial number, the manufacturing date, and the remaining amount of the sterilant, but the RF-ID of the cartridge 205 to be used a second or subsequent time has stored therein the serial number, the manufacturing date, the first use date and time, and the remaining amount of the sterilant. Accordingly, in step S101, the serial number, the manufacturing date, and the remaining amount of the sterilant are read from the RF-ID of the cartridge 205 to be first used while the serial number, the manufacturing date, the first use date and time, and the remaining amount of the sterilant are read from the RF-ID of the cartridge 205 to be used a second or subsequent time.

Thus, in step S102, it is determined that data has been read successfully from the RF-ID of the cartridge 205 to be first used when the serial number, the manufacturing date, and the remaining amount of the sterilant have been read successfully even in a case where the first use date and time has not been read from the RF-ID.

Subsequently, in step S102, in a case where it is determined that data has been read successfully from the RF-ID (YES in step S102), the sterilization apparatus 100 determines that the cartridge 205 is installed at the cartridge attaching position in the sterilization apparatus 100. In step S103, the sterilization apparatus 100 locks the cartridge attachment door 101. That is, locking is performed so that the cartridge 205 cannot be taken out. In this manner, in a case where data has been read successfully by the reading unit, locking is performed so that the cartridge 205 cannot be taken out.

Alternatively, for example, the cartridge 205 can be prevented from being taken out by preventing the injection needle to be inserted in the cartridge 205 from being pulled.

In other words, by inserting the injection needle in the cartridge 205 in step S103, extraction of the sterilant in the cartridge 205 is enabled, and the cartridge 205 can be prevented from being taken out.

In this manner, in a case where the cartridge 205 is attached to the cartridge attaching position in the sterilization apparatus 100, locking is performed so that the cartridge 205 cannot be taken out.

In a case where the cartridge 205 containing a remainder of the sterilant is attached to the cartridge attaching position in the sterilization apparatus 100, the sterilant can be prevented from being touched by the user since locking is performed so that the cartridge 205 cannot be taken out.

As described above, in a case where the cartridge 205 is installed in the sterilization apparatus 100, the sterilization apparatus 100 performs locking so that the cartridge 205 cannot be taken out. This is an example of application of a locking method according to the present exemplary embodiment.

Also, in a case where locking is released (the cartridge attachment door 101 is unlocked) in step S115 described below, locking is not performed, and the state in which locking is released in step S115 is kept in step S103.

Subsequently, in step S1031, the sterilization apparatus 100 determines whether the cartridge 205 is unsealed.

Specifically, for example, the unused cartridge 205 to be first used is not unsealed, and the RF-ID of the unused cartridge 205 does not have stored therein the first use date and time. Thus, since the first use date and time cannot be read from the RF-ID of the unused cartridge 205 in step S101, it is determined whether the cartridge 205 is unsealed by determining in step S1031 whether the first use date and time has been read successfully in step S101. In other words, in step S1031, it is determined that the cartridge 205 is unsealed in a case where the first use date and time has been read successfully in step S101 while it is determined that the cartridge 205 is not unsealed in a case where the first use date and time has not been read successfully in step S101.

The sterilization apparatus 100 advances the processing to step S104 in a case where it is determined in step S1031 that the cartridge 205 is not unsealed (NO in step S1031), while the sterilization apparatus 100 advances the processing to step S1032 in a case where it is determined in step S1031 that the cartridge 205 is unsealed (YES in step S1031).

In step S1032, the sterilization apparatus 100 moves the extraction needle 203-A to a predetermined position in the cartridge 205 in which the extraction needle 203-A does not dip in the sterilant.

More specifically, the arithmetic processing unit 201 of the sterilization apparatus 100 controls the extraction needle operation control unit 203 so that the extraction needle operation control unit 203 may move the extraction needle 203-A to a position in which the extraction needle 203-A is not pulled out of the cartridge 205 and in which the extraction needle 203-A does not dip in the sterilant in the cartridge 205 on condition that it is detected that the cartridge 205 has been attached to the sterilization apparatus 100.

In the present exemplary embodiment, the position in which the extraction needle 203-A is not pulled out of the cartridge 205 means a position in a case where the extraction needle 203-A has been pulled out from the cartridge 205.

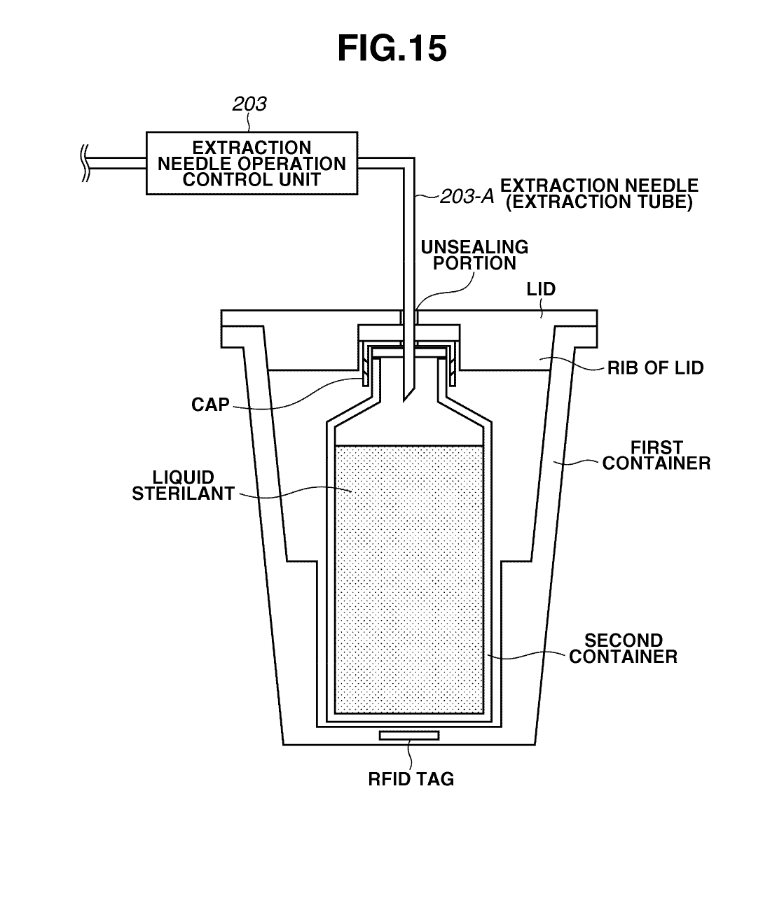

More specifically, as illustrated in FIG. 15, the extraction needle 203-A is moved so that the tip of the extraction needle 203-A may be located at a position (predetermined position) in which the extraction needle 203-A does not dip in the sterilant in the cartridge 205 and in which the extraction needle 203-A does not go out of the cartridge 205.

This operation of the extraction needle 203-A is performed by the extraction needle operation control unit 203.

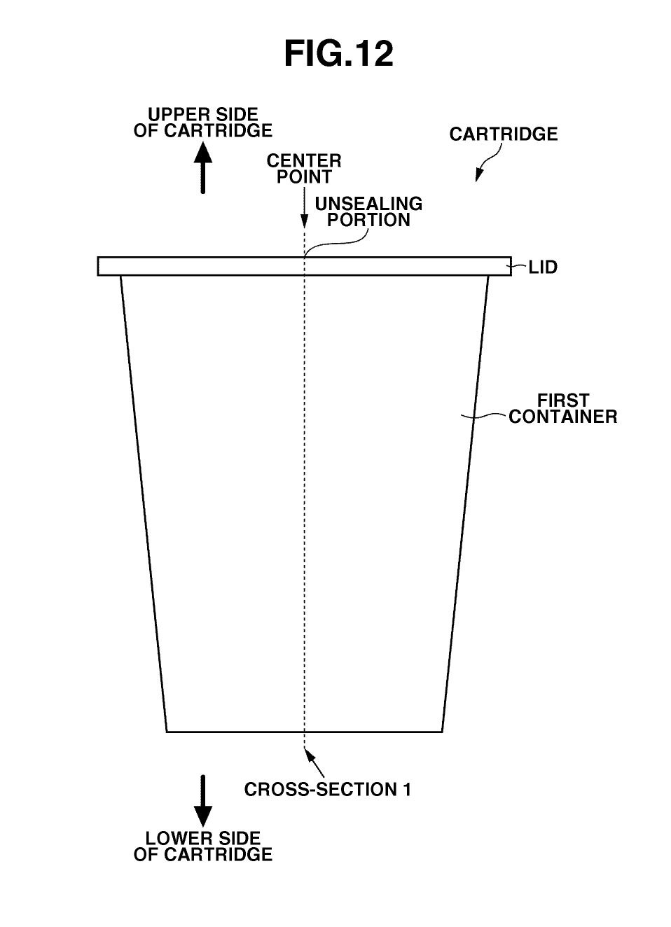

FIG. 15 is an example of a cross-sectional view along the cross-section 1 illustrating the cartridge 205 in FIG. 12 whose unsealing portion is sealed by the extraction needle 203-A that has been moved so that the tip of the extraction needle 203-A may be located at a position in the cartridge 205 in which the extraction needle 203-A does not dip in the sterilant.

As illustrated in FIG. 15, in the cartridge 205, a second container is filled with the liquid sterilant. The extraction needle 203-A is inserted into the cartridge 205 from the unsealing portion of the cartridge 205.

In step S1032, the extraction needle 203-A is moved to the lower side so that the tip of the extraction needle 203-A may not contact the liquid sterilant in this second container to seal the unsealing portion of the cartridge 205.

In step S1032, for example, in a case where the cartridge 205 containing the sterilant whose unsealing portion for inserting the extraction needle 203-A is unsealed is attached to the sterilization apparatus 100, the extraction needle 203-A is moved to the lower side, and is inserted in the unsealing portion to seal the unsealing portion of the cartridge 205.

As described above, since the extraction needle 203-A is moved to a position in which the extraction needle 203-A does not contact the liquid sterilant in the cartridge 205, the degree of acceleration of degradation of the liquid sterilant in the cartridge 205 can be delayed. Further, since the gasified sterilant is prevented from flowing in the sterilization apparatus 100, the degree of deterioration of respective parts in the sterilization apparatus 100 can be delayed.

After executing step S1032, the sterilization apparatus 100 advances the processing to step S104.

In step S104, the sterilization apparatus 100 determines whether the cartridge 205 contains a predetermined amount of the sterilant (for example, 8 ml) for one sterilization operation. More specifically, the sterilization apparatus 100 determines whether the remaining amount of the sterilant obtained from the RF-ID is more than the predetermined amount for one sterilization operation. Then, in a case where the sterilization apparatus 100 determines that the remaining amount of the sterilant is more than the predetermined amount for one sterilization operation, the sterilization apparatus 100 determines that the cartridge 205 contains the predetermined amount of the sterilant for one sterilization operation (sufficient sterilization processing can be executed) (YES in step S104) and performs processing in step S105. On the other hand, in a case where the sterilization apparatus 100 determines that the remaining amount of the sterilant is less than the predetermined amount (for example, 8 ml) for one sterilization operation, the sterilization apparatus 100 determines that the cartridge 205 does not contain the predetermined amount of the sterilant for one sterilization operation (sufficient sterilization processing cannot be executed) (NO in step S104) and performs processing in step S112.

In step S105, the sterilization apparatus 100 determines whether a predetermined period (for example, 13 months) has passed since the manufacturing date of the cartridge 205 obtained from the RF-ID.

In a case where the sterilization apparatus 100 determines that the predetermined period has passed from the manufacturing date (YES in step S105), the sterilization apparatus 100 determines that sufficient sterilization processing cannot be executed and performs processing in step S112. On the other hand, in a case where the sterilization apparatus 100 determines that the predetermined period has not passed since the manufacturing date (NO in step S105), the sterilization apparatus 100 determines that sufficient sterilization processing can be executed and performs processing in step S106.

In step S106, the sterilization apparatus 100 determines whether a predetermined period (for example, two weeks) has passed since the first use date and time obtained from the RF-ID.

For example, the first use date and time is not read from the RF-ID of the cartridge 205 to be first used in step S101. In this case, the sterilization apparatus 100 determines in step S106 that the predetermined period (for example, two weeks) has not passed since the first use date and time obtained from the RF-ID (NO in step S106).

In a case where the sterilization apparatus 100 determines that the predetermined period (for example, two weeks) has passed since the first use date and time obtained from the RF-ID (YES in step S106), the sterilization apparatus 100 determines that sufficient sterilization processing cannot be executed and performs processing in step S112. On the other hand, in a case where the sterilization apparatus 100 determines that the predetermined period (for example, two weeks) has not passed (NO in step S106), the sterilization apparatus 100 determines that sufficient sterilization processing can be executed and performs processing in step S107.

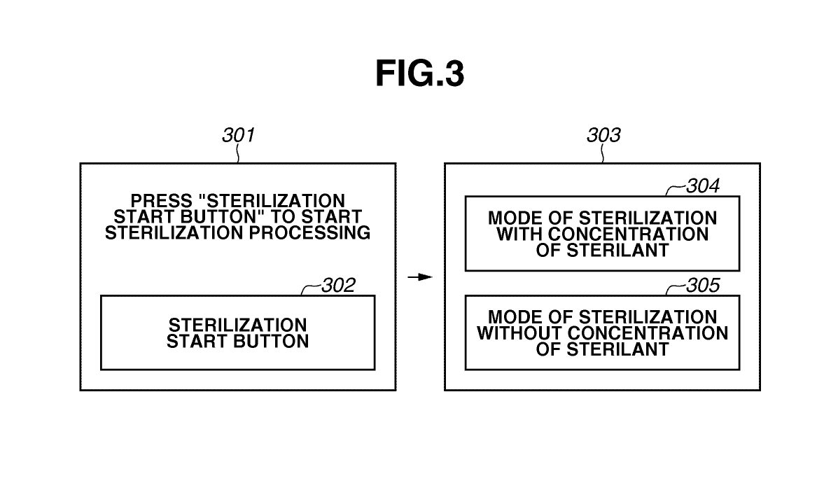

In step S107, the sterilization apparatus 100 displays a sterilization start screen (screen 301 in FIG. 3) on the display unit 102.

FIG. 3 illustrates an example of a screen to be displayed on the display unit 102 of the sterilization apparatus 100.

A “sterilization start button” 302 is displayed on the sterilization start screen 301. The “sterilization start button” 302 on the sterilization start screen 301 to be displayed in step S107 is ready to be pressed by the user (i.e., active).

In step S108, when the “sterilization start button” 302 is pressed by the user (YES in step S108), the sterilization apparatus 100 displays a sterilization mode selection screen (screen 303 in FIG. 3) on the display unit 102.

In step S109, a “mode of sterilization with concentration of sterilant” button 304 and a “mode of sterilization without concentration of sterilant” button 305 are displayed on the sterilization mode selection screen 303.

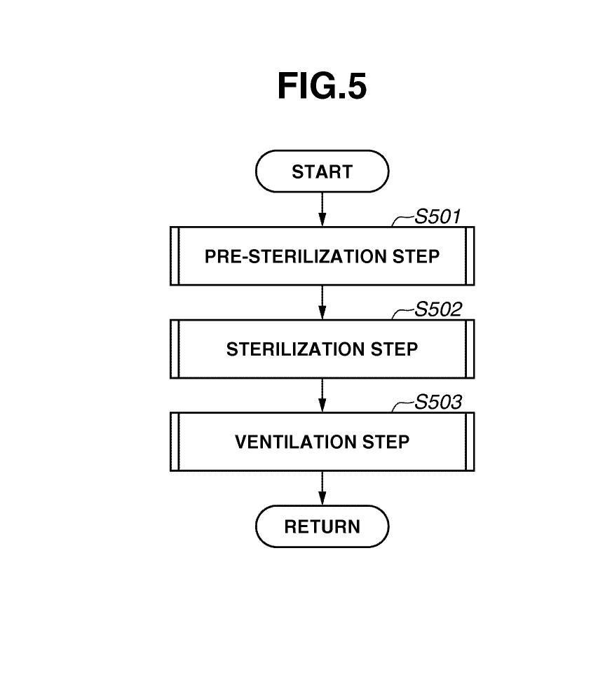

In step S110, the sterilization apparatus 100 accepts from the user selection of either the “mode of sterilization with concentration of sterilant” button 304 or the “mode of sterilization without concentration of sterilant” button 305. In step S111, the sterilization apparatus 100 performs sterilization processing according to the mode of the button selected by the user. Details of the sterilization processing in step S111 will be described below with reference to FIG. 5.

In this manner, the modes for the sterilization processing can be switched and used in one sterilization apparatus 100 according to an instruction by the user. More specifically, the sterilization processing is performed by concentrating the sterilant in a case where the “mode of sterilization with concentration of sterilant” button 304 is pressed by the user while the sterilization processing is performed by not concentrating the sterilant in a case where the “mode of sterilization without concentration of sterilant” button 305 is pressed by the user.

When the sterilization apparatus 100 ends the sterilization processing in step S111, the sterilization apparatus 100 returns the processing to step S101.

Also, in step S112, the sterilization apparatus 100 displays the sterilization start screen (screen 301 in FIG. 3) on the display unit 102. However, the “sterilization start button” 302 on the sterilization start screen (screen 301 in FIG. 3) to be displayed in step S112 is displayed so that the user cannot press the “sterilization start button” 302 (the “sterilization start button” 302 is inactive). Thus, an instruction for starting the sterilization processing by the user can be prevented from being accepted.

In step S113, the sterilization apparatus 100 determines from the serial number obtained from the RF-ID in step S101 whether the cartridge 205 installed at the cartridge attaching position is the cartridge 205 that has completed sterilant evacuation processing. More specifically, a memory (storage unit) in the sterilization apparatus 100 has stored therein the serial number enabling the sterilization apparatus 100 to identify the cartridge 205 that has completed the sterilant evacuation processing. By determining whether the serial number obtained from the RF-ID in step S101 corresponds to the serial number stored in the memory (storage unit), the sterilization apparatus 100 determines whether the cartridge 205 currently installed in the sterilization apparatus 100 is the cartridge 205 that has completed the sterilant evacuation processing.

Also, another example of determining whether the cartridge 205 is the cartridge 205 that has completed the sterilant evacuation processing will be described here.

When the sterilant evacuation processing in step S114 is completed, the sterilization apparatus 100 writes in the RF-ID of the cartridge 205 information indicating that the cartridge 205 has completed the sterilant evacuation processing.

In step S113, the sterilization apparatus 100 determines whether the information indicating that the cartridge 205 has completed the sterilant evacuation processing has been read successfully in step S101. The sterilization apparatus 100 advances the processing to step S115 in a case where the sterilization apparatus 100 determines that the information has been read successfully (YES in step S113) while the sterilization apparatus 100 advances the processing to step S114 in a case where the sterilization apparatus 100 determines that the information has not been read successfully (NO in step S113).

In this manner, the sterilization apparatus 100 can determine whether the cartridge 205 currently installed in the sterilization apparatus 100 is the cartridge 205 that has completed the sterilant evacuation processing.

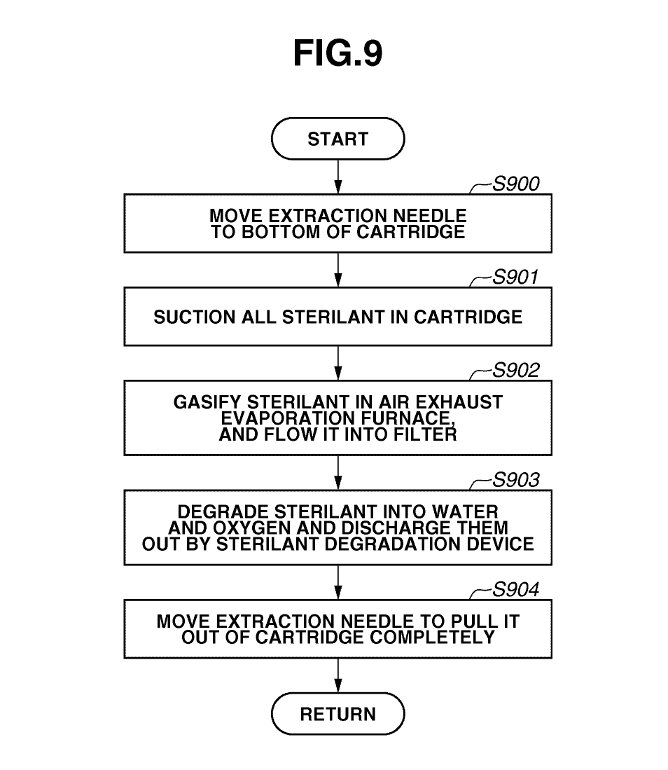

In a case where the sterilization apparatus 100 determines that the cartridge 205 currently installed in the sterilization apparatus 100 is the cartridge 205 that has completed the sterilant evacuation processing (YES in step S113), the sterilization apparatus 100 performs processing in step S115. On the other hand, in a case where the sterilization apparatus 100 determines that the cartridge 205 currently installed in the sterilization apparatus 100 is not the cartridge 205 that has completed the sterilant evacuation processing (NO in step S113), in step S114, the sterilization apparatus 100 performs the sterilant evacuation processing for suctioning, degrading, and discharging out of the sterilization apparatus 100 the entire amount of the liquid sterilant remaining in the cartridge 205, and thereafter performs processing in step S115. Details of the sterilant evacuation processing in step S114 will be described below with reference to FIG. 9.

Step S114 is an example of application of a disposal method according to the present exemplary embodiment disposing the hydrogen peroxide solution in the cartridge 205. That is, in the disposal method, all the sterilant (for example, a solution containing hydrogen peroxide) in the cartridge 205 is degraded with use of a catalyst (manganese dioxide) and is disposed of.

In a case where it is determined that the data read in step S101 satisfies predetermined conditions in step S104, step S105, and step S106, the sterilant in the cartridge 205 is disposed of by the disposal method.

More specifically, the predetermined conditions herein are a condition of whether the amount of the sterilant to be used for one sterilization processing operation remains in the cartridge 205, a condition of whether the predetermined period has passed since the manufacturing date of the cartridge 205, and a condition of whether the predetermined period has passed since the first use date and time of the cartridge 205.

When the processing in step S114 is performed, the serial number read in step S101 is stored in the memory (storage unit) in the sterilization apparatus 100 as a serial number enabling the sterilization apparatus 100 to identify the cartridge 205 that has completed the sterilant evacuation processing (disposal processing).

In step S115, the sterilization apparatus 100 unlocks the cartridge attachment door 101.

Step S115 is an example of application of an unlocking method according to the present exemplary embodiment releasing the locking by the locking method.

For example, the locking can be released by pulling out from the cartridge 205 the injection needle inserted in the cartridge 205.

In this manner, since the processing in step S114 for suctioning and disposing all the sterilant in the cartridge 205 is performed before releasing the locking, the sterilant can be prevented from being touched by the user, which improves safety.

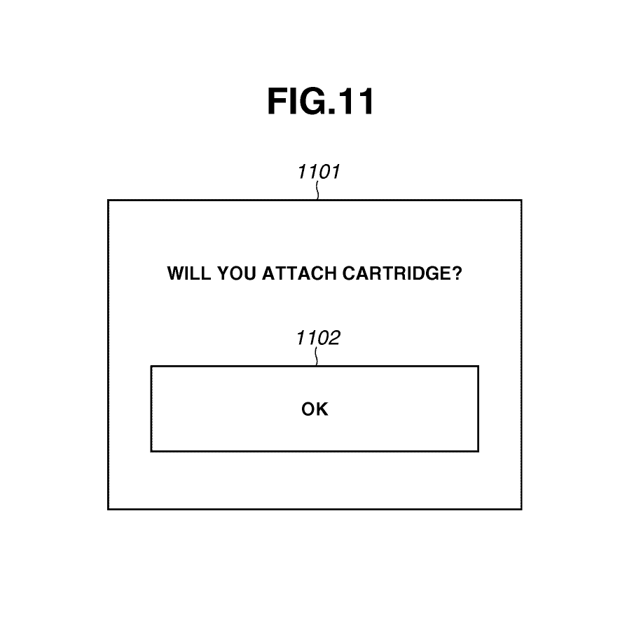

Also, in step S102, in a case where it is determined that data has not been read successfully from the RF-ID in step S101 (NO in step S102), the sterilization apparatus 100 determines that no cartridge 205 is installed at the cartridge attaching position in the sterilization apparatus 100. In step S116, the sterilization apparatus 100 displays a cartridge attachment request screen 1101 illustrated in FIG. 11.

FIG. 11 illustrates an example of the cartridge attachment request screen 1101 to be displayed on the display unit 102 of the sterilization apparatus 100.

On the cartridge attachment request screen 1101, an “OK” button 1102 is displayed.

In step S117, the sterilization apparatus 100 determines whether the “OK” button 1102 on the cartridge attachment request screen 1101 has been pressed by the user. In a case where the “OK” button 1102 is pressed (YES in step S117), in step S118, the sterilization apparatus 100 unlocks the cartridge attachment door 101 and returns the processing to step S101. On the other hand, in a case where the “OK” button 1102 is not pressed (NO in step S117), the sterilization apparatus 100 keeps displaying the cartridge attachment request screen 1101.

Unlocking and locking of the cartridge attachment door 101 are performed by operations of the lock operation control unit 202.

Next, an example of detailed processing of the sterilization processing illustrated in step S111 in FIG. 4 will be described with reference to FIG. 5.

FIG. 5 illustrates an example of detailed processing of the sterilization processing performed in step S111 in FIG. 4.

Each step (process) illustrated in FIG. 5 is performed by control of operations of each device in the sterilization apparatus 100 by the arithmetic processing unit 201 of the sterilization apparatus 100.

In other words, the arithmetic processing unit 201 of the sterilization apparatus 100 executes a program readable and executable by the arithmetic processing unit 201 of the sterilization apparatus 100 to control operations of each device and execute each step (process) illustrated in FIG. 5.

When a step performed in step S501 in FIG. 5 is started, all the valves in the sterilization apparatus 100 (valve (V1) 211, valve (V2) 215, valve (V3) 212, valve (V4) 213, valve (V9) 227, and valve (V7) 226) are in closed states.

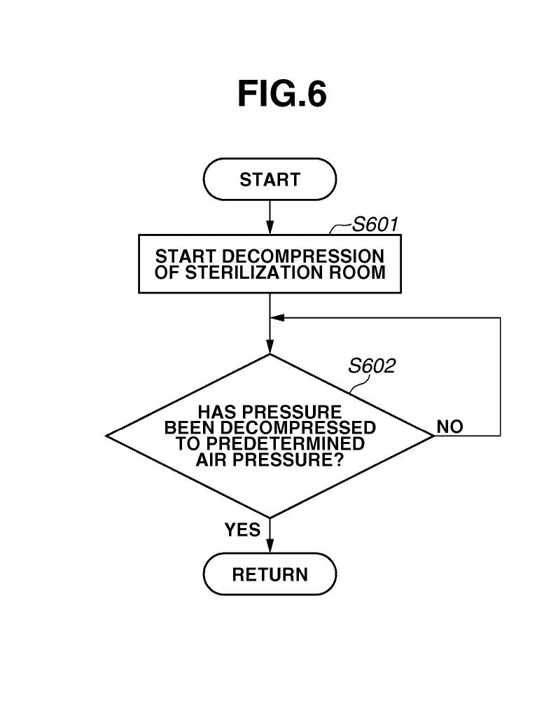

First, in step S501, the sterilization apparatus 100 performs a pre-sterilization step for operating the air feeding vacuum pump 220, suctioning gas in the sterilization room. 219, and performing decompression until the air pressure in the sterilization room 219 reaches a predetermined air pressure (for example, 45 Pa). Detailed processing of the processing in the pre-sterilization step will be described below with reference to FIG. 6.

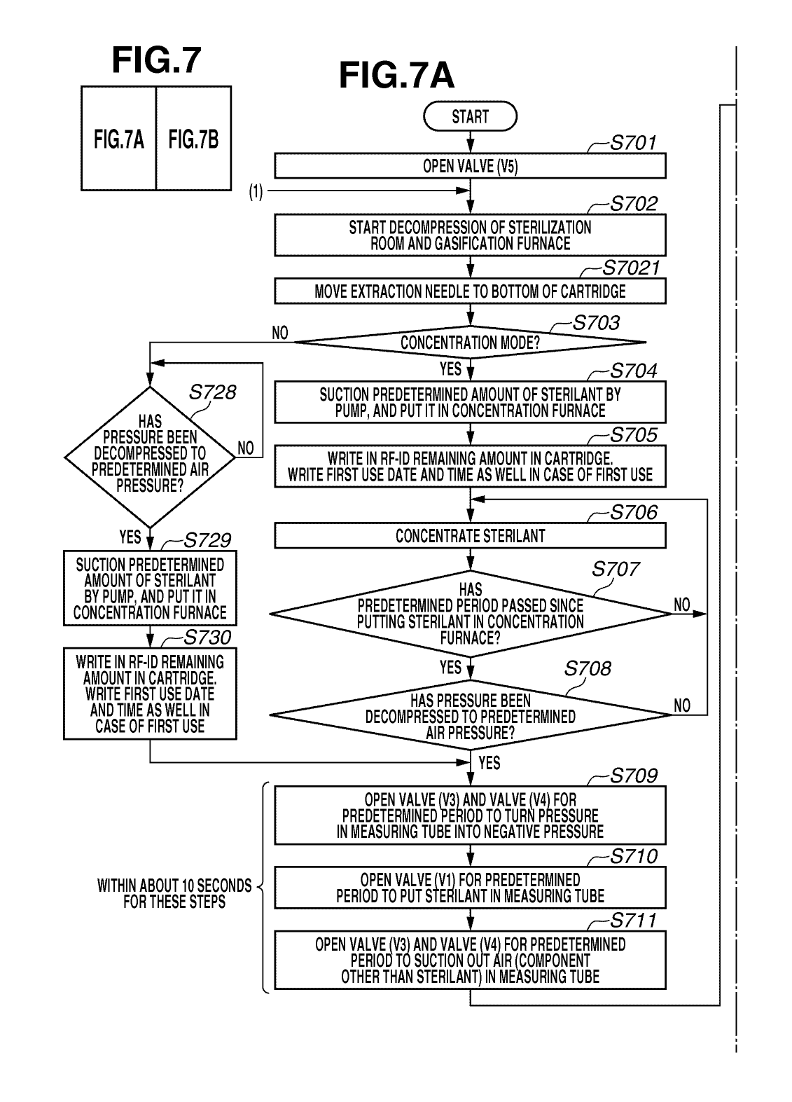

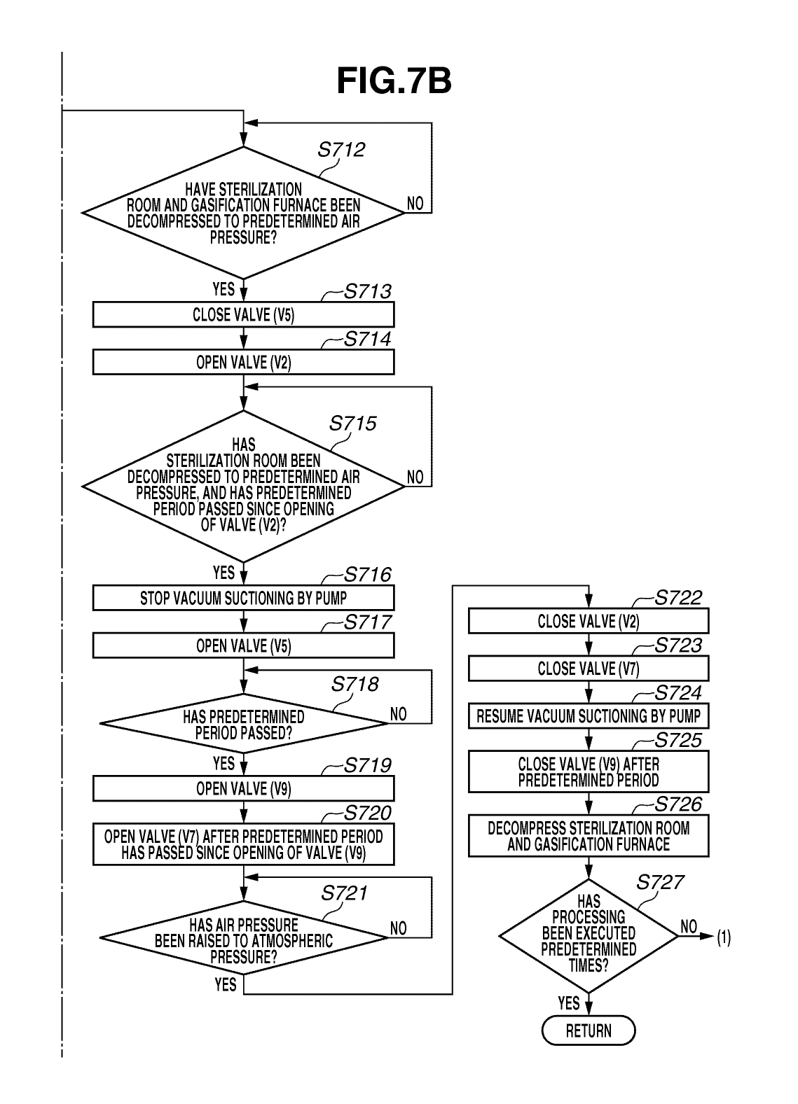

Subsequently, in step S502, the sterilization apparatus 100 performs a sterilization step for putting the sterilant in the sterilization room 219 and sterilizing a sterilization target. Detailed processing of the processing performed in the sterilization step will be described below with reference to FIG. 7.

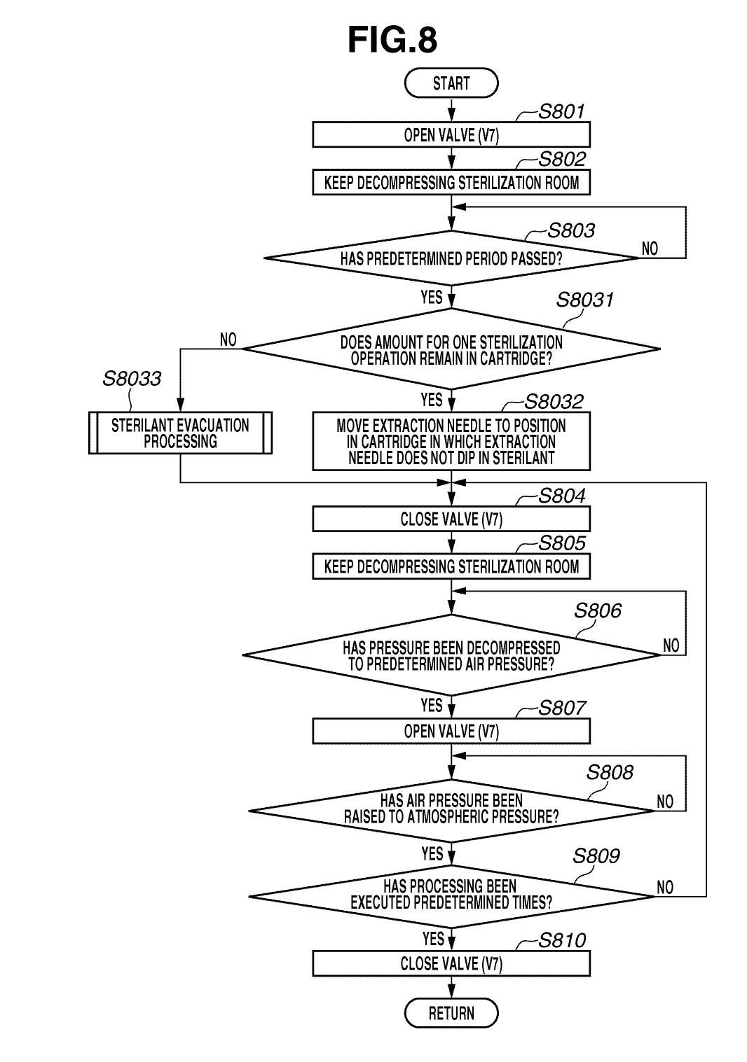

Subsequently, in step S503, the sterilization apparatus 100 performs a ventilation step for eliminating the sterilant contained in the sterilization room 219 and the gasification furnace 216. Detailed processing for the processing in the ventilation step will be described below with reference to FIG. 8.

Next, an example of detailed processing in the pre-sterilization step performed in step S501 in FIG. 5 will be described with reference to FIG. 6.

FIG. 6 illustrates an example of detailed processing in the pre-sterilization step illustrated in step S501 in FIG. 5.

Each step (process) illustrated in FIG. 6 is performed by controlling operations of each device in the sterilization apparatus 100 by the arithmetic processing unit 201 of the sterilization apparatus 100.

In other words, the arithmetic processing unit 201 of the sterilization apparatus 100 executes a program readable and executable by the arithmetic processing unit 201 of the sterilization apparatus 100 to control operations of each device and execute each step (process) illustrated in the figure.

First, in step S601, the sterilization apparatus 100 starts processing for operating the air feeding vacuum pump 220 and suctioning gas in the sterilization room 219.

In step S602, the sterilization apparatus 100 determines whether the pressure (air pressure) in the sterilization room 219 has been decompressed to a predetermined air pressure (for example, 45 Pa). More specifically, the sterilization apparatus 100 determines whether the pressure (air pressure) in the sterilization room 219 measured by the pressure sensor provided in the sterilization room 219 has been decompressed to the predetermined air pressure (for example, 45 Pa).

In step S602, in a case where it is determined that the pressure (air pressure) in the sterilization room 219 has not been decompressed to the predetermined air pressure (for example, 45 Pa) (NO in step S602), the sterilization apparatus 100 keeps operating the air feeding vacuum pump 220, suctions gas in the sterilization room 219, and reduces the pressure (air pressure) in the sterilization room 219.

On the other hand, in step S602, in a case where it is determined that the pressure (air pressure) in the sterilization room 219 has been decompressed to the predetermined air pressure (for example, 45 Pa) (YES in step S602), the sterilization apparatus 100 keeps operating the air feeding vacuum pump 220, suctions gas in the sterilization room 219, and starts processing in step S502.

Next, an example of detailed processing in the sterilization step performed in step S502 in FIG. 5 will be described with reference to FIG. 7.

FIG. 7 (composed of FIGS. 7A and 7B) illustrates an example of detailed processing in the sterilization step performed in step S502 in FIG. 5.

Each step (process) illustrated in FIG. 7 is performed by controlling operations of each device in the sterilization apparatus 100 by the arithmetic processing unit 201 of the sterilization apparatus 100.

In other words, the arithmetic processing unit 201 of the sterilization apparatus 100 executes a program readable and executable by the arithmetic processing unit 201 of the sterilization apparatus 100 to control operations of each device and execute each step (process) illustrated in the figure.

First, in step S701, the sterilization apparatus 100 opens the valve (V5) 217, and thereby the sterilization room 219 and the gasification furnace 216 communicate by the duct. With this operation, since gas in the sterilization room 219 is currently being suctioned by the air feeding vacuum pump 220 for decompression, in step S702, decompression in the sterilization room 219 and the gasification furnace 216 is started.

Subsequently, in step S7021, the sterilization apparatus 100 moves the extraction needle 203-A to the lower side, so that the tip of the extraction needle 203-A may be located at or near a bottom of the cartridge 205.

That is, to extract the sterilant in the cartridge 205 by using the extraction needle 203-A, the arithmetic processing unit 201 controls the extraction needle operation control unit 203 so that the extraction needle 203-A may move to the bottom or the proximity of the bottom in the cartridge 205.

As a result, the extraction needle operation control unit 203 (movement unit) moves the extraction tube to a position for extracting the sterilant in the cartridge by the extraction tube in a case of extracting the sterilant in the cartridge by the extraction tube.

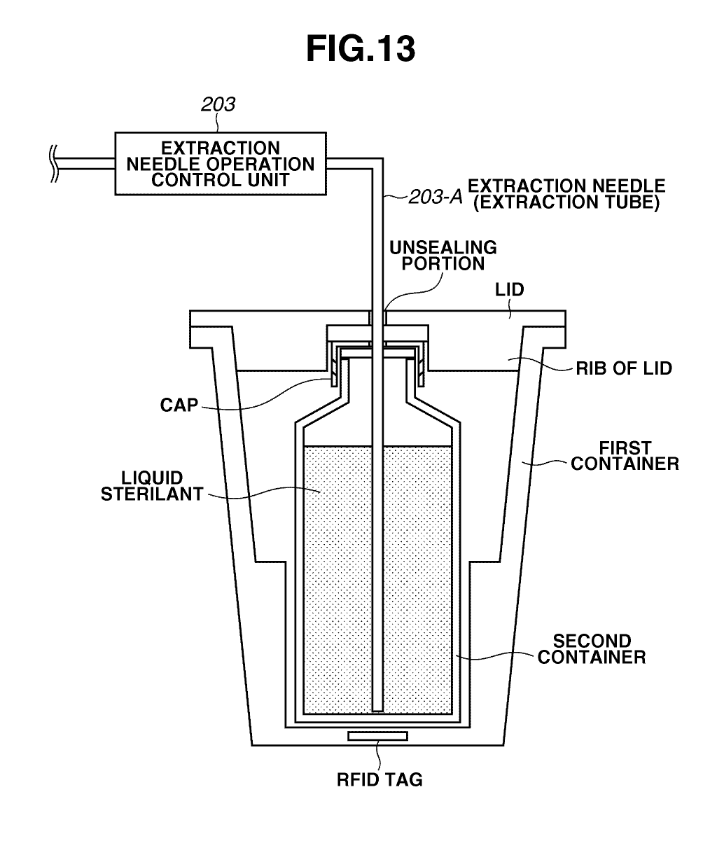

More specifically, as illustrated in FIG. 13, the arithmetic processing unit 201 moves the extraction needle 203-A by operating the extraction needle operation control unit 203 so that the tip of the extraction needle 203-A may be located at a predetermined position that is the bottom or the proximity of the bottom of the cartridge 205.

In this manner, by moving the extraction needle 203-A to the bottom or the vicinity of the bottom of the cartridge 205, the liquid sterilant in the cartridge 205 can be extracted in step S729 or step S704.

Subsequently, in step S703, the sterilization apparatus 100 determines which button has been pressed in step S110, i.e., the “mode of sterilization with concentration of sterilant” button 304 or the “mode of sterilization without concentration of sterilant” button 305. The sterilization apparatus 100 performs processing in step S704 in a case where it is determined that the “mode of sterilization with concentration of sterilant” button 304 has been pressed (YES in step S703) while the sterilization apparatus 100 performs processing in step S728 in a case where it is determined that the “mode of sterilization without concentration of sterilant” button 305 has been pressed (NO in step S703).

Here, the case in which the “mode of sterilization with concentration of sterilant” button 304 has been pressed (case in which the sterilization processing is performed by concentrating the sterilant) will first be described.

In step S704, the sterilization apparatus 100 operates the liquid feeding rotary pump 207 and suctions a predetermined amount (for example, 2 ml) of the sterilant in the cartridge 205. The sterilization apparatus 100 then puts the predetermined amount of the suctioned sterilant in the concentration furnace 208. Here, the predetermined amount of the suctioned sterilant is an amount enabling the space in the sterilization room 219 to be put into a saturated state by the sterilant, for example.

In step S705, the sterilization apparatus 100 writes a remaining amount of the sterilant in the cartridge 205, in the RF-ID of the cartridge 205 attached to the cartridge attaching position. More specifically, the sterilization apparatus 100 stores in the RF-ID a value derived by subtracting the predetermined amount (for example, 2 ml) suctioned from the cartridge 205 in step S704 from the remaining amount of the sterilant in the cartridge 205 read in step S101.

That is, in step S705, the sterilization apparatus 100 stores in the RF-ID a value derived by subtracting a total sum of an amount suctioned from the cartridge 205 in step S704 from the remaining amount of the sterilant in the cartridge 205 read in step S101.

Also, in step S705, the sterilization apparatus 100 determines that the cartridge 205 is first used in the sterilization apparatus 100 this time in a case where information indicating date and time is not contained in the first use date and time (date and time when the cartridge 205 is first used in the sterilization apparatus 100) read from the RF-ID in step S101. In other words, the sterilization apparatus 100 determines that the cartridge 205 is first used in the sterilization apparatus 100 this time in a case where the first use date and time has not been read successfully from the RF-ID in step S101.

As described above, only in a case where it is determined that the cartridge 205 is first used in the sterilization apparatus 100, the sterilization apparatus 100 writes current date and time information in the RF-ID as well.

The sterilization apparatus 100 keeps the heater provided in the concentration furnace 208 heated while the sterilization apparatus 100 is powered. Thus, in step S706, the sterilant put in the concentration furnace 208 in step S704 is heated by the heat of the heater to cause water contained in the sterilant in the concentration furnace 208 to be evaporated.

The reason for keeping the heater provided in the concentration furnace 208 heated while the sterilization apparatus 100 is powered is to enable the sterilization apparatus 100 to be used immediately all the time in a surgery room, for example. Thus, by eliminating time required to heat the heater in the concentration furnace 208, the sterilization apparatus 100 can be used immediately all the time.

Here, specifically, the heater provided in the concentration furnace 208 is warmed at 80° C., for example, in a case where the sterilant is hydrogen peroxide liquid (also referred to as a hydrogen peroxide solution). Through this operation, water can be evaporated (gasified) mainly, and the sterilant can be concentrated.

Subsequently, in step S707, the sterilization apparatus 100 determines whether a predetermined period (for example, 6 minutes) has passed since putting the sterilant in the concentration furnace 208 in step S704. In a case where it is determined that the predetermined period has passed since putting the sterilant in the concentration furnace 208 (YES in step S707), the sterilization apparatus 100 performs processing in step S708. On the other hand, in a case where the predetermined period has not passed since putting the sterilant in the concentration furnace 208 (NO in step S707), the sterilization apparatus 100 leaves the sterilant in the concentration furnace 208 and keeps concentrating the sterilant.

Subsequently, in step S708, the sterilization apparatus 100 determines whether the air pressure in the sterilization room 219 and the gasification furnace 216 has been decompressed to a predetermined air pressure (for example, 500 Pa).

In a case where the air pressure in the sterilization room 219 and the gasification furnace 216 has been decompressed to the predetermined air pressure (YES in step S708), in step S709, the sterilization apparatus 100 opens the valve (V3) 212 and the valve (V4) 213 for a predetermined period (opens the valve (V3) 212 and the valve (V4) 213 for a predetermined period (for example, 3 seconds) and closes the valve (V3) 212 and the valve (V4) 213) to decompress the pressure in the measuring tube 214. On the other hand, in a case where the air pressure in the sterilization room 219 and the gasification furnace 216 has not been decompressed to the predetermined air pressure (NO in step S708), the sterilization apparatus 100 keeps concentrating the sterilant.

Subsequently, in step S710, when the sterilization apparatus 100 opens the valve (V1) 211 for a predetermined period (for example, 3 seconds) after opening the valve (V3) 212 and the valve (V4) 213 for the predetermined period and then closing the valve (V3) 212 and the valve (V4) 213 in step S709, the sterilant contained in the concentration furnace 208 is suctioned and goes into the measuring tube 214 since the air pressure in the measuring tube 214 is lower than the air pressure in the concentration furnace 208 (outside). Here, by opening the valve (V1) 211 for the predetermined period and then closing the valve (V1) 211, the sterilant contained in the concentration furnace 208 is suctioned and goes into the measuring tube 214. Here, not only the sterilant but also air in the concentration furnace 208 is suctioned into the measuring tube 214.

Thereafter, the pressure in the sterilization room 219 is kept decompressed by the air feeding vacuum pump 220 continuously.

Thus, the air pressure in the sterilization room 219 becomes lower than the air pressure in the measuring tube 214. More specifically, the air pressure in the sterilization room 219 is about 400 Pa while the air pressure in the measuring tube 214 is about the atmospheric pressure (101325 Pa). The reason why the air pressure in the measuring tube 214 is raised close to the atmospheric pressure is that not only the sterilant but also air in the concentration furnace 208 are suctioned into the measuring tube 214.

Subsequently, in step S711, the sterilization apparatus 100 opens the valve (V3) 212 and valve (V4) 213 for a predetermined period (for example, 3 seconds) to suction out air (not containing the liquid sterilant) in the measuring tube 214 to the sterilization room 219. In this step, when the predetermined period has passed since opening the valve (V3) 212 and valve (V4) 213, the sterilization apparatus 100 closes the valve (V3) 212 and valve (V4) 213.

Subsequently, in step S712, the sterilization apparatus 100 determines whether the air pressure in the sterilization room 219 and the gasification furnace 216 has been decompressed to a predetermined air pressure (for example, 80 Pa). In a case where it is determined that the air pressure has been decompressed (YES in step S712), in step S713, the sterilization apparatus 100 closes the valve (V5) 217.

In step S714, the sterilization apparatus 100 opens the valve (V2) 215. Through this operation, the sterilant in the measuring tube 214 is suctioned into the gasification furnace 216 and is gasified in the gasification furnace 216.

Here, the sterilant is gasified in the gasification furnace 216 as a molecular cluster.

The sterilization room 219 has a larger volume than that of the gasification furnace 216, and in the gasification furnace 216, the sterilant is gasified as the molecular cluster. The reason for this is that, since the volume of the gasification furnace 216 is smaller than that of the sterilization room 219, each distance between the molecules of the sterilant in the gasification furnace 216 is short, and the molecular cluster is easily formed by an intermolecular force.

At this time, the air feeding vacuum pump 220 keeps suctioning gas in the sterilization room. 219 and decompressing the pressure in the sterilization room 219 continuously. A air pressure in the gasification furnace 216 into which the sterilant in the measuring tube 214 has been suctioned is raised.

Thus, the air pressure in the gasification furnace 216 is higher than the air pressure in the sterilization room 219.

Subsequently, in step S715, the sterilization apparatus 100 determines whether the air pressure in the sterilization room 219 has been decompressed to a predetermined air pressure (for example, 50 Pa) and whether a predetermined period has passed since opening the valve (V2) 215 in step S714. In a case where the air pressure in the sterilization room 219 has been decompressed to the predetermined air pressure (for example, 50 Pa) and where the predetermined period has passed since opening the valve (V2) 215 in step S714 (YES in step S715), in step S716, the sterilization apparatus 100 stops suctioning (vacuum suction) in the sterilization room 219 by the air feeding vacuum pump 220, and in step S717, the sterilization apparatus 100 opens the valve (V5) 217. Through this operation, the gasified sterilant is diffused in the sterilization room 219, which enables the sterilization target to be sterilized.

The reason for the diffusion is that the air pressure in the sterilization room 219 (for example, 50 Pa) is lower than the air pressure in the gasification furnace 216.

The sterilant diffused here is one into which the molecular cluster in the gasification furnace 216 is further segmentalized, and can further be diffused in the sterilization room 219, which enables enhancement of a sterilization effect.

Also, small lumens of the sterilization target can be sterilized effectively.

In step S718, the sterilization apparatus 100 determines whether a predetermined period (for example, 330 seconds) has passed since opening the valve (V5) 217 in step S717. In a case where it is determined that the predetermined period (for example, 330 seconds) has passed since opening the valve (V5) 217 (YES in step S718), in step S719, the sterilization apparatus 100 opens the valve (V9) 227.

Through this operation, since the air pressure in the gasification furnace 216 and the sterilization room 219 is lower than the air pressure outside the sterilization apparatus 100, external air (air) outside the sterilization apparatus 100 cleaned by the air intake HEPA filter 210 is suctioned into the gasification furnace 216. By the air fed into the gasification furnace 216, the gaseous sterilant filled in the gasification furnace 216 and the sterilant attached to an inner surface of the gasification furnace 216 are fed into the sterilization room 219, which enhances a sterilization effect on the sterilization target in the sterilization room 219. Thus, a sterilization effect on a part that is difficult to sterilize such as a back of a thin tube of the sterilization target is enhanced, for example.

In step S720, when a predetermined period (15 seconds) has passed since opening the valve (V9) 227 in step S719, the sterilization apparatus 100 opens the valve (V7) 226, and external air (air) outside the sterilization apparatus 100 cleaned by the air intake HEPA filter 210 is further suctioned into the sterilization room 219. The reason why the external air (air) outside the sterilization apparatus 100 is suctioned into the sterilization room 219 is that the air pressure in the sterilization room 219 and the gasification furnace 216 is lower than the air pressure outside the sterilization apparatus 100.

Through this operation, a sterilization effect on a part that is difficult to sterilize such as a back of a thin tube of the sterilization target (especially, a lumen part) is enhanced.

Subsequently, in step S721, the sterilization apparatus 100 determines whether the air pressure in the sterilization room 219 and the gasification furnace 216 has been raised to the atmospheric pressure. In a case where it is determined that the air pressure has been raised to the atmospheric pressure (YES in step S721), in step S722, the sterilization apparatus 100 closes the valve (V2) 215.

Subsequently, in step S723, the sterilization apparatus 100 closes the valve (V7) 226, and in step S724, the sterilization apparatus 100 resumes suctioning (vacuum suctioning) in the sterilization room 219 by the air feeding vacuum pump 220. Through this operation, external air (air) outside the sterilization apparatus 100 cleaned by the air intake HEPA filter 210 is suctioned into the gasification furnace 216 via the duct by which the air intake HEPA filter 210 and the gasification furnace 216 communicate with each other. By the air fed into the gasification furnace 216, the gaseous sterilant filled in the gasification furnace 216 and the sterilant attached to the inner surface of the gasification furnace 216 are further fed into the sterilization room 219.

Thus, a sterilization effect on a part that is difficult to sterilize such as a back of a thin tube of the sterilization target (especially, a lumen part) is enhanced, and the amount of the sterilant in the gasification furnace 216 can be reduced effectively.

In step S725, after a predetermined period (for example, 15 seconds) has passed since the resumption of suctioning (vacuum suction) in the sterilization room 219 by the air feeding vacuum pump 220 in step S724, the sterilization apparatus 100 closes the valve (V9) 227.

At this time, the sterilization apparatus 100 keeps suctioning (vacuum suctioning) in the sterilization room 219 by the air feeding vacuum pump 220 continuously, the sterilization room 219 and the gasification furnace 216 are sealed in step S725, and in step S726, the sterilization apparatus 100 decompresses the pressure in the sterilization room 219 and the gasification furnace 216.

Subsequently, in step S727, the sterilization apparatus 100 determines whether the processing from step S702 to step S726 has been executed predetermined times (for example, four times), and in a case where it is determined that the processing has been executed (YES in step S727), the sterilization apparatus 100 performs the processing in step S503. On the other hand, in a case where it is determined that the processing from step S702 to step S726 has not been executed predetermined times (NO in step S727), the sterilization apparatus 100 performs step S702 and the subsequent processing again. As described above, by executing the processing from step S702 to step S726 predetermined times, a sterilization effect on the sterilization target is enhanced, and the sterilization target can be sterilized sufficiently.

Next, the case in which it is determined that the “mode of sterilization without concentration of sterilant” button 305 has been pressed in step S703 (case in which the sterilization processing is performed by not concentrating the sterilant) will be described.

In a case where it is determined that the “mode of sterilization without concentration of sterilant” button 305 has been pressed in step S703 (NO in step S703), then in step S728, the sterilization apparatus 100 determines whether the air pressure in the sterilization room 219 and the gasification furnace 216 has been decompressed to a predetermined air pressure (for example, 1000 Pa).

In a case where it is determined that the air pressure in the sterilization room 219 and the gasification furnace 216 has been decompressed to the predetermined air pressure (for example, 1000 Pa) (YES in step S728), then in step S729, the sterilization apparatus 100 operates the liquid feeding rotary pump 207 and suctions a predetermined amount (for example, 2 ml) of the sterilant in the cartridge 205. The sterilization apparatus 100 then puts the predetermined amount of the suctioned sterilant in the concentration furnace 208.

Here, the predetermined amount of the suctioned sterilant is an amount enabling the space in the sterilization room 219 to be put into a saturated state by the sterilant, for example.

Subsequently, in step S730, the sterilization apparatus 100 writes a remaining amount of the sterilant in the cartridge 205 in the RF-ID of the cartridge 205 attached to the cartridge attaching position. More specifically, the sterilization apparatus 100 stores in the RF-ID a value obtained by subtracting the predetermined amount (for example, 2 ml) suctioned from the cartridge 205 in step S729 from the remaining amount of the sterilant in the cartridge 205 read in step S101.

In a case where a predetermined amount of the sterilant per suction suctioned from the cartridge 205 is 2 ml, for example, where it is determined in step S727 that the processing has not been executed predetermined times in step S727 (NO in step S727), and where the number of times of performing the processing in step S702 and the following steps is two, a total sum of an amount of the sterilant suctioned from the cartridge 205 in step S729 is (2 ml (predetermined amount)×2 times=) 4 ml. Thus, in step S730, the sterilization apparatus 100 stores in the RF-ID a value derived by subtracting 4 ml, which is a total sum of an amount of the sterilant suctioned from the cartridge 205 in step S729 from the remaining amount of the sterilant in the cartridge 205 read in step S101.

That is, in step S730, the sterilization apparatus 100 stores in the RF-ID a value derived by subtracting a total sum of an amount of the sterilant suctioned from the cartridge 205 in step S729 from the remaining amount of the sterilant in the cartridge 205 read in step S101.

Also, in step S730, the sterilization apparatus 100 determines that the cartridge 205 is first used in the sterilization apparatus 100 this time in a case where information indicating date and time is not contained in the first use date and time (date and time when the cartridge 205 is first used in the sterilization apparatus 100) read from the RF-ID in step S101. Thus, the sterilization apparatus 100 determines that the cartridge 205 is first used in the sterilization apparatus 100 this time in a case where the first use date and time has not been read successfully in step S101.

In this manner, only in a case where it is determined that the cartridge 205 is first used in the sterilization apparatus 100, the sterilization apparatus 100 writes current date and time information in the RF-ID.

After the sterilization apparatus 100 performs the processing in step S730, the sterilization apparatus 100 performs the aforementioned processing in step S709 and the subsequent steps.

When the air pressure in the sterilization room 219 reaches a predetermined air pressure (for example, 1000 Pa) in step S728, the sterilization apparatus 100 starts suctioning the sterilant in step S729, and the air pressure in the sterilization room 219 becomes lower than 500 Pa when the sterilization apparatus 100 finishes suctioning the sterilant in step S729. Accordingly, the sterilization apparatus 100 can advance the processing to step S709 efficiently.

As described above, after the air pressure in the sterilization room 219 and the gasification furnace 216 has been decompressed to the predetermined air pressure (for example, 1000 Pa), which is an air pressure when decompression in the measuring tube 214 is started, the predetermined amount of the suctioned sterilant is put in the concentration furnace 208, the pressure in the measuring tube 214 can be decompressed in step S709 immediately, and thereafter the sterilant in the concentration furnace 208 is put in the measuring tube 214 in step S710. Thus, the sterilant can be put in the measuring tube 214 from the concentration furnace 208 immediately. Thus, the sterilant can be put in the measuring tube 214 with almost no concentration of the sterilant in the concentration furnace 208.

Next, an example of detailed processing of the ventilation processing illustrated in step S503 in FIG. 5 will be described with reference to FIG. 8.

FIG. 8 illustrates an example of detailed processing in the ventilation step performed in step S503 in FIG. 5.

Each step (process) illustrated in FIG. 8 is performed by controlling operations of each device in the sterilization apparatus 100 by the arithmetic processing unit 201 of the sterilization apparatus 100.

That is, the arithmetic processing unit 201 of the sterilization apparatus 100 executes a program readable and executable by the arithmetic processing unit 201 of the sterilization apparatus 100 to control operations of each device and execute each step (process) illustrated in the figure.

First, in step S801, the sterilization apparatus 100 opens the valve (V7) 226.

In step S802, the sterilization apparatus 100 keeps suctioning (vacuum suctioning) in the sterilization room 219 by the air feeding vacuum pump 220 continuously.

In step S803, when a predetermined period has passed (YES in step S803) since opening the valve (V7) 226 in step S801 and suctioning (vacuum suctioning) in the sterilization room 219 by the air feeding vacuum pump 220 in step S802, the sterilization apparatus 100 starts executing processing in step S8031 and executes processing in step S8033 or processing in step S8032.

Also, when the predetermined period has passed (YES in step S803), processing in step S804 and the subsequent steps is executed in parallel with the processing in step S8031 and the processing in step S8033 or the processing in step S8032.

Although execution of the processing in step S8031 and the processing in step S8033 or the processing in step S8032 after the predetermined period has passed (YES in step S803) will be described here, the processing in step S8031 and the processing in step S8033 or the processing in step S8032 may be executed between step S704 and step S705. In this case, processing in step S705 is executed after execution of the processing in step S8033 or the processing in step S8032. Similarly, the processing in step S8031 and the processing in step S8033 or the processing in step S8032 may be executed between step S729 and step S730. In this case, processing in step S730 is executed after executing the processing in step S8033 or the processing in step S8032.

Here, the processing in step S8031 and the processing in step S8033 or the processing in step S8032 will be described.

In step S8031, the sterilization apparatus 100 determines whether the cartridge 205 contains an amount of the sterilant for one sterilization operation.

Specifically, the sterilization apparatus 100 determines whether the cartridge 205 contains an amount of the sterilant for one sterilization operation according to a value to be stored in the RF-ID of the cartridge 205 in step S730 or step S705.

In step S730, the sterilization apparatus 100 stores in the RF-ID as an amount of the sterilant remaining in the cartridge 205 a value obtained by subtracting the total sum of the amount of the sterilant suctioned from the cartridge 205 in step S729 from the remaining amount of the sterilant in the cartridge 205 read in step S101.

Also, in step S705, the sterilization apparatus 100 stores in the RF-ID as an amount of the sterilant remaining in the cartridge 205 a value obtained by subtracting the total sum of the amount of the sterilant suctioned from the cartridge 205 in step S704 from the remaining amount of the sterilant in the cartridge 205 read in step S101.

Thus, the sterilization apparatus 100 determines whether the amount (remaining amount) of the sterilant in the cartridge 205 to be stored in the RF-ID of the cartridge 205 in step S730 or step S705 is the predetermined amount (for example, 8 ml) of the sterilant for one sterilization operation or more.

In this manner, the sterilization apparatus 100 can determine whether the amount (remaining amount) of the sterilant in the cartridge 205 is the predetermined amount (for example, 8 ml) of the sterilant for one sterilization operation or more, but other examples of determination will also be described.

For example, a weight sensor for measuring weight of the cartridge 205 is provided at a cartridge setting position, the weight sensor measures (detects) weight of the cartridge 205 after the sterilant has been extracted from the cartridge 205 in step S704 or step S729, and the sterilization apparatus 100 determines whether the measured weight (value) is a predetermined value or more. The sterilization apparatus 100 determines that the amount (remaining amount) of the sterilant in the cartridge 205 is the predetermined amount of the sterilant for one sterilization operation or more in a case where it is determined that the measured weight (value) is the predetermined value or more. On the other hand, the sterilization apparatus 100 determines that the amount (remaining amount) of the sterilant in the cartridge 205 is less than the predetermined amount of the sterilant for one sterilization operation in a case where it is determined that the measured weight (value) is less than the predetermined value.

Also, the sterilization apparatus 100 can determine whether the cartridge 205 contains the predetermined amount of the sterilant for one sterilization operation by adopting other conventional techniques such as a method for detecting the remaining amount of the sterilant in the cartridge 205 by using an optical sensor such as an infrared sensor.

In a case where it is determined that the amount (remaining amount) of the sterilant in the cartridge 205 is the predetermined amount (for example, 8 ml) of the sterilant for one sterilization operation or more (YES in step S8031), the sterilization apparatus 100 advances the processing to step S8032. On the other hand, in a case where it is determined that the amount (remaining amount) of the sterilant in the cartridge 205 is less than the predetermined amount (for example, 8 ml) of the sterilant for one sterilization operation (NO in step S8031), the sterilization apparatus 100 advances the processing to step S8033.

Step S8031 is an example of application of a determination method according to the present exemplary embodiment, and based on the amount of the sterilant extracted by using the extraction needle 203-A, the number of times of extraction of the sterilant, or the weight of the cartridge 205 from which the sterilant has been extracted (the result of extraction of the sterilant from the cartridge by the extraction tube), the sterilization apparatus 100 determines whether the cartridge after extraction of the sterilant contains the predetermined amount of the sterilant required for the sterilization processing. On condition that it is determined that the cartridge 205 contains the predetermined amount of the sterilant required for the sterilization processing (YES in step S8031), the sterilization apparatus 100 executes the processing in step S8032.

Also, on condition that it is determined that the cartridge 205 does not contain the predetermined amount of the sterilant required for the sterilization processing (NO in step S8031), the sterilization apparatus 100 executes the processing in step S8033.

In step S8032, the sterilization apparatus 100 moves the extraction needle 203-A to a predetermined position in the cartridge 205 in which the extraction needle 203-A does not dip in the sterilant.

In step S8032, the sterilization apparatus 100 moves the extraction needle 203-A so that the tip of the extraction needle 203-A may be located at a similar position to the position to which the extraction needle 203-A moves in step S1032 in FIG. 4.

More specifically, as illustrated in FIG. 15, the sterilization apparatus 100 moves the extraction needle 203-A so that the tip of the extraction needle 203-A may be located at a position (predetermined position) in which the extraction needle 203-A does not dip in the sterilant in the cartridge 205 and in which the extraction needle 203-A does not go out of the cartridge 205.

In step S8032, the sterilization apparatus 100 moves the extraction needle 203-A to an upper side so that the extraction needle 203-A may be located at the predetermined position in which the tip of the extraction needle 203-A does not contact the liquid sterilant in the second container of the cartridge 205 and seals the unsealing portion of the cartridge 205.

In this manner, the arithmetic processing unit 201 executes the processing in step S8032 for controlling the extraction needle operation control unit 203 so that the extraction needle operation control unit 203 may move the extraction needle 203-A to a position in which the extraction needle 203-A is not pulled out of the cartridge 205 and in which the extraction needle 203-A does not dip in the sterilant in the cartridge 205 after the sterilant has been extracted from the cartridge 205 with use of the extraction needle 203-A, thus to enable the degree of acceleration of degradation of the liquid sterilant in the cartridge 205 to be delayed.

That is, since the extraction needle 203-A is moved to a position in which the extraction needle 203-A does not contact the liquid sterilant in the cartridge 205, the degree of acceleration of degradation of the liquid sterilant in the cartridge 205 can be delayed. Further, since the gasified sterilant is prevented from flowing in the sterilization apparatus 100, the degree of deterioration of respective parts in the sterilization apparatus 100 can be delayed.

That is, as described above in the description of step S8032, in a case where the extraction needle operation control unit 203 (movement unit) of the sterilization apparatus 100 waits for extraction of the sterilant from the cartridge by the extraction tube to perform the following sterilization processing with the same cartridge, the extraction needle operation control unit 203 (movement unit) moves the extraction tube in an opposite direction (opposite direction of an extraction tube inserting direction) of a moving direction of the extraction tube moving for extraction of the sterilant in the cartridge.

In step S8032, the extraction needle operation control unit 203 (movement unit) moves the extraction tube so that the extraction tube may be located at a position in which the extraction tube is not pulled out of the cartridge and in which the extraction tube does not dip in the sterilant in the cartridge on condition that the sterilization processing has been executed predetermined times in step S727, and that the predetermined amount of the sterilant to be used for the sterilization processing has been extracted from the cartridge in step S704 or step S729.

As described above, the extraction needle operation control unit 203 (movement unit) moves the extraction tube so that a position of the extraction tube with respect to the cartridge in a case of extracting the sterilant in the cartridge by the extraction tube (position in step S7021) and a position of the extraction tube (position in step S8032) with respect to the cartridge in a case of waiting for extraction of the sterilant from the cartridge by the extraction tube to perform the following sterilization processing with the same cartridge may differ from each other.

Step S8033 is the same processing to that performed in step S114. In step S8033, the sterilization apparatus 100 performs the sterilant evacuation processing for suctioning, degrading, and discharging out of the sterilization apparatus 100 the entire amount of the liquid sterilant remaining in the cartridge 205.

Step S8033 or the sterilant degradation device 222 is an example of application of a disposal method or unit according to the present exemplary embodiment for disposing the hydrogen peroxide solution in the cartridge 205. In the disposal method or unit, all the sterilant (for example, a solution containing hydrogen peroxide) in the cartridge 205 is degraded with use of a catalyst (manganese dioxide) and is disposed of.

In step S8033 or the sterilant degradation device 222, the sterilant extracted from the cartridge 205 is disposed of.

Details of the sterilant evacuation processing in step S8033 will be described below with reference to FIG. 9.

When the processing in step S8033 is performed, the serial number read in step S101 is stored in the memory (storage unit) in the sterilization apparatus 100 as a serial number enabling the sterilization apparatus 100 to identify the cartridge 205 that has completed the sterilant evacuation processing (disposal processing).

In this manner, the processing in step S8031 and the processing in step S8033 or the processing in step S8032 end, which have been being executed in parallel with the processing in step S804 and the following steps.

Next, the processing in step S804 and the subsequent steps will be described.

In step S804, the sterilization apparatus 100 closes the valve (V7) 226, and in step S805, the sterilization apparatus 100 keeps suctioning (vacuum suctioning) in the sterilization room 219 by the air feeding vacuum pump 220 continuously. Through this operation, the pressure in the sterilization room 219 is decompressed.

Subsequently, in step S806, when the pressure in the sterilization room 219 has been decompressed to the predetermined air pressure (50 Pa) (YES in step S806), then in step S807, the sterilization apparatus 100 opens the valve (V7) 226. Through this operation, external air (air) outside the sterilization apparatus 100 cleaned by the air intake HEPA filter 210 is suctioned into the sterilization room 219. The reason why the external air (air) outside the sterilization apparatus 100 is suctioned into the sterilization room 219 is that the air pressure in the sterilization room 219 is lower than the air pressure outside the sterilization apparatus 100.

In step S808, the sterilization apparatus 100 determines whether the air pressure in the sterilization room 219 has been raised to the atmospheric pressure. In a case where it is determined that the air pressure has been raised to the atmospheric pressure (YES in step S808), then in step S809, the sterilization apparatus 100 determines whether the processing from step S804 to step S808 has been executed predetermined times (for example, four times), and in a case where the processing from step S804 to step S808 has been executed predetermined times (for example, four times) (YES in step S809), in step S810, the sterilization apparatus 100 closes the valve (V7) 226 to end the ventilation step.

On the other hand, in a case where the processing from step S804 to step S808 has not been executed predetermined times (for example, four times) (NO in step S809), the sterilization apparatus 100 performs the processing from step S804 again.

Through this operation, the sterilant attached to a surface of the sterilization room 219 and the gaseous sterilant remaining in the sterilization room 219 are suctioned by the air feeding vacuum pump 220. Gas (containing the sterilant) suctioned here passes through the air exhaust HEPA filter 221, the sterilant is degraded in the sterilant degradation device 222, and degraded molecules are discharged outside.

Next, an example of detailed processing for the sterilant evacuation processing performed in step S114 in FIG. 4 and in step S8033 in FIG. 8 will be described with reference to FIG. 9.

FIG. 9 illustrates an example of detailed processing for the sterilant evacuation processing performed in step S114 in FIG. 4 and in step S8033 in FIG. 8.

Each step (process) illustrated in FIG. 9 is performed by controlling operations of each device in the sterilization apparatus 100 by the arithmetic processing unit 201 of the sterilization apparatus 100.

In other words, the arithmetic processing unit 201 of the sterilization apparatus 100 executes a program readable and executable by the arithmetic processing unit 201 of the sterilization apparatus 100 to control operations of each device and execute each step (process) illustrated in the figure.

First, in step S900, the sterilization apparatus 100 operates the extraction needle operation control unit 203 to move the extraction needle 203-A so that the tip of the extraction needle 203-A may be located at or near the bottom of the cartridge 205.

In other words, to extract the sterilant in the cartridge 205 by using the extraction needle 203-A, the arithmetic processing unit 201 controls the extraction needle operation control unit 203 so that the extraction needle 203-A may move to the bottom or the proximity of the bottom in the cartridge 205.

More specifically, as illustrated in FIG. 13, the arithmetic processing unit 201 moves the extraction needle 203-A by operating the extraction needle operation control unit 203 so that the tip of the extraction needle 203-A may be located at a predetermined position that is the bottom or the proximity of the bottom of the cartridge 205.

In a case where the tip of the extraction needle 203-A is not inserted in the cartridge 205 at a stage immediately before execution of step S900, the extraction needle 203-A is moved to the lower side until the tip of the extraction needle 203-A reaches the predetermined position (i.e., the bottom or the proximity of the bottom of the cartridge 205). At this time, the unsealing portion of the cartridge 205 comes into a penetrating state by the extraction needle 203-A.

Also, in a case where the tip of the extraction needle 203-A is located at the position (predetermined position) in which the extraction needle 203-A does not dip in the sterilant in the cartridge 205 and in which the extraction needle 203-A does not go out of the cartridge 205 at a stage immediately before execution of step S900 as illustrated in FIG. 15, the extraction needle 203-A is moved to the lower side until the tip of the extraction needle 203-A reaches the predetermined position which is the bottom or the proximity of the bottom of the cartridge 205.

Also, in a case where the tip of the extraction needle 203-A is already located at the predetermined position (i.e., the bottom or the proximity of the bottom of the cartridge 205) at a stage immediately before execution of step S900, the state is kept.

In this manner, by moving the extraction needle 203-A to the bottom or the proximity of the bottom of the cartridge 205, the liquid sterilant in the cartridge 205 is ready to be extracted in step S901.

Subsequently, in step S901, by the liquid feeding rotary pump 223, the sterilization apparatus 100 suctions all the liquid sterilant in the cartridge 205 by the pump and feeds all the sterilant fed through the duct between the liquid sensor 204 and the liquid feeding rotary pump 223 to the air exhaust evaporation furnace 224 through the duct between the liquid feeding rotary pump 223 and the air exhaust evaporation furnace 224.

In step S902, by the air exhaust evaporation furnace 224, the sterilization apparatus 100 heats by the heater installed in the air exhaust evaporation furnace 224 all the liquid sterilant (sterilant accumulated in the air exhaust evaporation furnace 224) to be fed through the duct between the liquid feeding rotary pump 223 and the air exhaust evaporation furnace 224 and gasifies all the sterilant. Subsequently, the gasified sterilant is fed to the air exhaust HEPA filter 221 through the duct between the air exhaust HEPA filter 221 and the air exhaust evaporation furnace 224.

Here, the heater installed in the air exhaust evaporation furnace 224 is heated at a higher temperature than a boiling point of the sterilant (hydrogen peroxide) (the boiling point of hydrogen peroxide is 141° C.), for example. Accordingly, all the sterilant will be gasified by the air exhaust evaporation furnace 224.

By the air exhaust HEPA filter 221, the sterilization apparatus 100 cleans the gasified sterilant fed through the duct between the air exhaust evaporation furnace 224 and the air exhaust HEPA filter 221, and the cleaned gas (containing the sterilant) passes through the duct between the sterilant degradation device 222 and the air exhaust HEPA filter 221 and is fed to the sterilant degradation device 222.

In step S903, the sterilant degradation device 222 degrades molecules of the sterilant contained in the gas fed through the duct between the sterilant degradation device 222 and the air exhaust HEPA filter 221 and discharges the molecules generated by degradation out of the sterilization apparatus 100.

Subsequently, in step S904, in a case where the sterilization apparatus 100 feeds all the liquid sterilant in the cartridge 205 to the air exhaust evaporation furnace 224 in step S901, the sterilization apparatus 100 operates the extraction needle operation control unit 203 to move the extraction needle 203-A inserted in the cartridge 205 to be pulled out of the cartridge 205.

In this manner, to dispose of the sterilant contained in the cartridge by the disposal unit in step S8033 (FIG. 9) on condition that it is determined in step S8031 that the cartridge does not contain the predetermined amount of the sterilant required for the sterilization processing (NO in step S8031), the extraction needle operation control unit 203 (movement unit) moves the extraction tube to the bottom of the cartridge, and in step S901, the sterilant in the cartridge is extracted by using the moved extraction tube. In step S904, on condition that the sterilant has been extracted, the extraction needle operation control unit 203 (movement unit) moves the extraction tube to pull the extraction tube out of the cartridge.

In other words, the arithmetic processing unit 201 controls the extraction needle operation control unit 203 so as to pull the extraction needle 203-A out of the cartridge 205 on condition that all the sterilant in the cartridge 205 has been extracted by using the extraction needle 203-A to dispose of the sterilant contained in the cartridge 205 in step S901.

In this manner, the extraction needle 203-A is moved to the upper side and is pulled out of the cartridge 205. Through this operation, locking can be released.

Next, a block configuration of the hardware configuration of the concentration furnace 208, the valve (V1) 211, the valve (V3) 212, the valve (V4) 213, the measuring tube 214, the valve (V2) 215, the gasification furnace 216, the valve (V5) 217, and the valve (V9) 227 in the sterilization apparatus 100 according to the present exemplary embodiment will be described with reference to FIG. 10.

FIG. 10 illustrates an example of a block configuration diagram for the hardware configuration of the concentration furnace 208, the valve (V1) 211, the valve (V3) 212, the valve (V4) 213, the measuring tube 214, the valve (V2) 215, the gasification furnace 216, the valve (V5) 217, and the valve (V9) 227 in the sterilization apparatus 100 according to the present exemplary embodiment.

As for the respective hardware components illustrated in FIG. 10, identical components to those in FIG. 2 are designated with the same reference numerals.

In step S704 and step S729, the liquid feeding rotary pump 207 is operated to suction a predetermined amount (for example, 2 ml) of the sterilant in the cartridge 205 and put the predetermined amount of the suctioned sterilant in the concentration furnace 208.

As illustrated in FIG. 10, the concentration furnace 208 is provided at a lower portion thereof with the heater, and in step S706, the sterilant is heated by heat of this heater. In a case where the sterilant is a hydrogen peroxide solution, water is gasified by the heat of this heater. The gasified water is pushed into the duct communicating with the air exhaust HEPA filter 221 by air fed from the air feeding pressure pump 209 via the duct and is exhausted from the concentration furnace 208. Through this operation, the sterilant (hydrogen peroxide solution) is concentrated.

As described referring to FIG. 7, in step S710, the sterilant in the concentration furnace 208 is put into the measuring tube 214.

This measuring tube 214 includes a straight tube portion 1001 and a branch tube portion 1002 as illustrated in FIG. 10.

The straight tube portion 1001 is a straight tubular portion. The tube of the straight tube portion 1001 is arranged in a gravity direction.

Also, the branch tube portion 1002 is a tubular portion extending in a branch shape from a middle portion or an upper portion of the straight tube portion 1001.

The straight tube portion 1001 is installed so that an axis of the straight tube portion 1001 and an axis of the branch tube portion 1002 may be perpendicular to each other.

With such a configuration, the sterilant coming from the concentration furnace 208 accumulates in the straight tube portion 1001 in the measuring tube 214. A portion of the straight tube portion 1001 in which the sterilant accumulates is referred to as sterilant accumulation portion 1003.

Thus, the sterilant accumulation portion 1003 has a sufficient space to accommodate the sterilant coming from the concentration furnace 208.

Therefore, the sterilant coming from the concentration furnace 208 accumulates in the sterilant accumulation portion 1003, and air coming from the concentration furnace 208 together with the sterilant is filled in a space other than the space for the sterilant accumulating in the sterilant accumulation portion 1003. As a result, since the space other than the space for the sterilant is also a space in the branch tube portion 1002 and a space communicating with the space in the branch tube portion 1002, the air is suctioned into the sterilization room 219 by opening of the valve (V3) 212 and the valve (V4) 213 in step S711.

When the valve (V2) 215 is opened in step S714, the sterilant accumulating in the sterilant accumulation portion 1003 is suctioned into the gasification furnace 216 and is gasified. As illustrated in FIG. 10, the sterilant is easily gasified since the liquid sterilant goes into the gasification furnace 216 from an upper portion of the gasification furnace 216.

Also, the duct between the air intake HEPA filter 210 and the gasification furnace 216 is provided at the upper portion of the gasification furnace 216 as illustrated in FIG. 10. Accordingly, when the valve (V9) 227 is opened in step S719, air (external air) flows from the upper portion of the gasification furnace 216 to the sterilization room 219 located at a lower portion of the gasification furnace 216, and thus the sterilant attached to the inside of the gasification furnace 216 and the gasified sterilant in the gasification furnace 216 are easily removed over a wide range, and the more removed sterilant can be fed into the sterilization room 219.

Next, the cartridge 205 and a state in which the extraction needle 203-A has been inserted in the cartridge 205 will be described with reference to FIGS. 12 and 13.

FIG. 12 is a side view of the cartridge 205 for the sterilant to be used in the sterilization apparatus 100 according to the present exemplary embodiment.

The cartridge 205 illustrated in FIG. 12 is a cartridge containing as much sterilant as doses for several sterilization processing operations in one bottle.

The cartridge 205 illustrated in FIG. 12 contains solution containing hydrogen peroxide to be used as the sterilant.

As illustrated in FIG. 12, the cartridge 205 includes a first container and a lid of the first container.

As for the outer appearance, the first container is formed in a glass shape. In addition, a material for the first container is polypropylene (plastic) resistant to hydrogen peroxide as the sterilant. This first container is provided to protect the second container described below.

The lid is provided on an upper side of the first container to close the first container. Therefore, the lid adheres to a rim of an outer circumference of the first container. A material for this lid is polypropylene (plastic) resistant to hydrogen peroxide as the sterilant.

A cross-section of the cartridge 205 at a center point of the cartridge 205 as seen from the upper side of the cartridge 205 is a cross-section 1.

Next, an inside structure of the cartridge 205 when the tip of the extraction needle 203-A has been inserted to the bottom or the proximity of the bottom of the cartridge 205 to suction the sterilant in the cartridge 205 will be described with reference to FIG. 13.

FIG. 13 is a cross-sectional view along the cross-section 1 of the cartridge 205 when the tip of the extraction needle 203-A has been inserted to the bottom or the proximity of the bottom of the cartridge 205 to suction the sterilant in the cartridge 205.

The sterilization apparatus 100 operates the extraction needle 203-A (injection needle) to lower the extraction needle 203-A toward the cartridge 205 from the upper portion (upper side) to the lower portion (lower side) of the cartridge 205 to cause the extraction needle 203-A (injection needle) to be inserted in a hole of the lid and a hole of a cap (unsealing portion).

At this time, the sterilization apparatus 100 operates the injection needle so that the injection needle may pass through the hole of the lid and the hold of the cap and so that the tip of the injection needle may reach the lower portion of the second container.

As illustrated in FIG. 13, in step S103, by inserting the extraction needle 203-A to the bottom or the proximity of the bottom of the cartridge 205, the sterilant in the cartridge 205 can be extracted, and the cartridge 205 can be prevented from being taken out.

FIG. 15 illustrates an example of a cross-sectional view along the cross-section 1 of the cartridge 205 whose unsealing portion is sealed by the extraction needle 203-A that has been moved to a position in the cartridge 205 in which the tip of the extraction needle 203-A does not dip in the sterilant.

In FIG. 15, the extraction needle 203-A is moved further to the upper side than that in FIG. 13 so that the tip of the extraction needle 203-A may be located at the position in which the tip of the extraction needle 203-A does not dip in the sterilant.

Thus, since the unsealing portion of the cartridge is sealed, substances in the atmosphere (such as dust) are prevented from entering the cartridge. In addition, since the extraction tube does not contact the sterilant, the degree of acceleration of degradation of the liquid sterilant in the cartridge can be delayed. Further, since sealing of the unsealing portion of the cartridge prevents the gasified sterilant from flowing in the sterilization apparatus, the degree of deterioration of respective parts in the sterilization apparatus can be delayed.

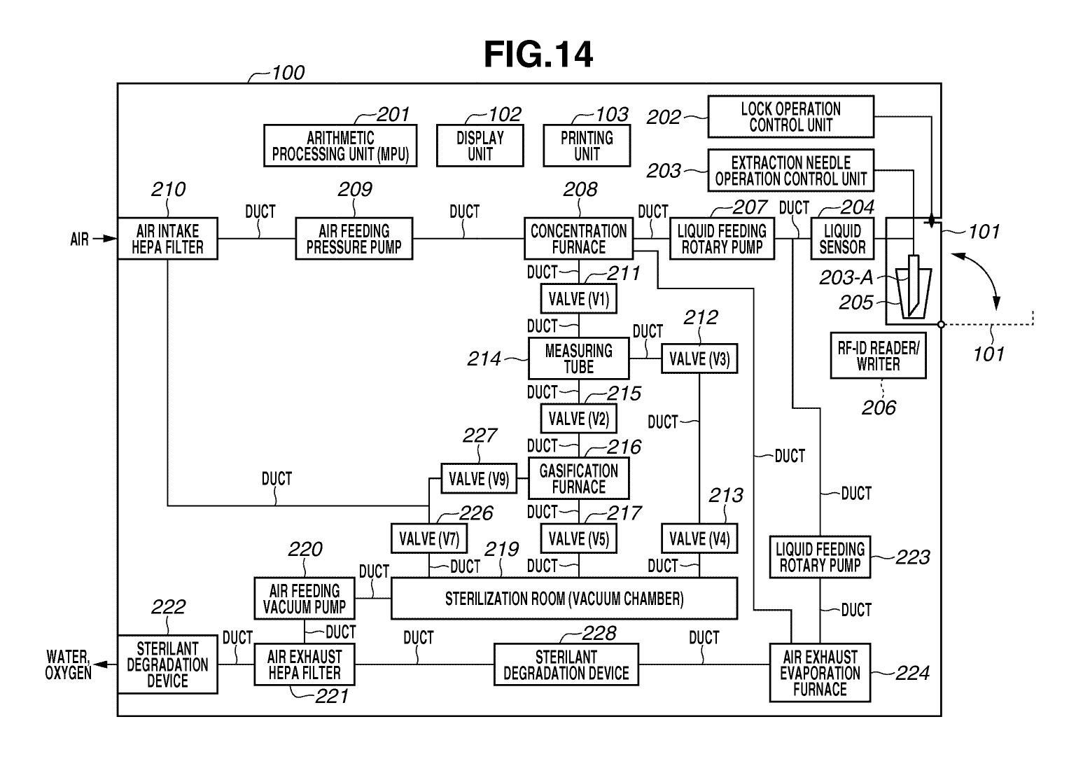

A sterilization apparatus according to a second exemplary embodiment will be described with reference to FIG. 14.

As for the second exemplary embodiment, different portions from those in the sterilization apparatus described in the present exemplary embodiment are described.

FIG. 14 illustrates an example of a hardware configuration of the sterilization apparatus according to the present exemplary embodiment.

Although the duct enabling direct communication between the concentration furnace 208 and the air exhaust HEPA filter 221 is provided in the sterilization apparatus 100 according to the first exemplary embodiment, the duct enabling direct communication between the concentration furnace 208 and the air exhaust HEPA filter 221 is not provided in the present exemplary embodiment.

Thus, the sterilization apparatus 100 according to the present exemplary embodiment is provided with a duct enabling communication between the concentration furnace 208 and the air exhaust evaporation furnace 224 as illustrated in FIG. 14.

Further, although the duct enabling direct communication between the air exhaust evaporation furnace 224 and the air exhaust HEPA filter 221 is provided in the sterilization apparatus 100 according to the first exemplary embodiment, sterilant degradation device 228 is newly provided between the air exhaust evaporation furnace 224 and the air exhaust HEPA filter 221 in the present exemplary embodiment.

The sterilant degradation device 228 is an example of application of a disposal unit according to the present exemplary embodiment for disposing of the sterilant contained in the cartridge 205 attached to the sterilization apparatus 100.

As illustrated in FIG. 14, the sterilization apparatus 100 according to the present exemplary embodiment is provided with the sterilant degradation device 228 between the air exhaust evaporation furnace 224 and the air exhaust HEPA filter 221, and is provided with a duct enabling communication between the air exhaust evaporation furnace 224 and the sterilant degradation device 228 and a duct enabling communication between the sterilant degradation device 228 and the air exhaust HEPA filter 221.

The sterilization apparatus 100 according to the present exemplary embodiment is similar to the sterilization apparatus 100 according to the first exemplary embodiment other than the aforementioned configuration.

Since the sterilization apparatus 100 according to the present exemplary embodiment is configured as illustrated in FIG. 14, the sterilization apparatus 100 according to the present exemplary embodiment is controlled in the following manner.

The concentration furnace 208 heats by the heater the sterilant fed from the liquid feeding rotary pump 207 via the duct and evaporates (gasifies) water contained in the sterilant to concentrate the sterilant.

The gasified water is pushed into the duct communicating with the air exhaust evaporation furnace 224 from the concentration furnace 208 by air fed from the air feeding pressure pump 209 via the duct, and is exhausted from the concentration furnace 208.

Gas and/or liquid (this liquid is liquid into which gas gasified in the concentration furnace 208 is condensed in the duct allowing direct communication between the concentration furnace 208 and the air exhaust evaporation furnace 224) coming from the concentration furnace 208, passing through the duct allowing direct communication between the concentration furnace 208 and the air exhaust evaporation furnace 224, and going into the air exhaust evaporation furnace 224 are/is heated again by the heater in the air exhaust evaporation furnace 224, and the gas reaches a higher temperature and is difficult to condense. Further, the condensed liquid is heated again by the heater in the air exhaust evaporation furnace 224, and is thus gasified. The heated gas and/or the gasified gas are/is fed from the air exhaust evaporation furnace 224 via the duct allowing direct communication between the air exhaust evaporation furnace 224 and the sterilant degradation device 228 to the sterilant degradation device 228.

Similar to the sterilant degradation device 222, the sterilant degradation device 228 is provided with a catalyst for degrading the sterilant.

Thus, when the gasified sterilant is fed from the air exhaust evaporation furnace 224 to the sterilant degradation device 228, the catalyst and the sterilant react with each other, and the sterilant is degraded.

In a case where the sterilant is hydrogen peroxide, for example, the catalyst that degrades the sterilant is manganese dioxide, for example. In this case, in the sterilant degradation device 228, hydrogen peroxide reacts with manganese dioxide, and is degraded into water and oxygen.

Further, since the reaction in which hydrogen peroxide is degraded into water and oxygen is a heat generating reaction, since water and oxygen have lower boiling points than that of hydrogen peroxide, the gas containing hydrogen peroxide is degraded into water and oxygen in the sterilant degradation device 228, is further heated, and is converted into gas in a state of being difficult to condense.

Gas containing water (gaseous water) and oxygen (gaseous oxygen) generated by degradation in the sterilant degradation device 228 passes through the duct allowing direct communication between the sterilant degradation device 228 and the air exhaust HEPA filter 221, and is fed to the air exhaust HEPA filter 221.

Here, since the gas (for example, water and oxygen) fed to the air exhaust HEPA filter 221 has a lower boiling point, is at a higher temperature, and is more difficult to condense than the gas (for example, hydrogen peroxide) fed from the air exhaust evaporation furnace 224 to the sterilant degradation device 228, the gas fed to the air exhaust HEPA filter 221 is in a state of being difficult to condense in the air exhaust HEPA filter 221. Accordingly, liquefied liquid is not attached to the air exhaust HEPA filter 221 easily.

The air exhaust HEPA filter 221 may become extremely poor in air permeability and may not function normally when the air exhaust HEPA filter 221 absorbs liquid.

For example, in a case where the air feeding pressure pump 209 or the air feeding vacuum pump 220 is operated in a state where the air exhaust HEPA filter 221 absorbs liquid, the air exhaust HEPA filter 221 may be clogged and may not function normally.

To solve such a problem, the sterilization apparatus 100 according to the present exemplary embodiment newly includes the sterilant degradation device 228 between the air exhaust evaporation furnace 224 and the air exhaust HEPA filter 221.

With this configuration, liquefied liquid is not attached to the air exhaust HEPA filter 221 easily, and the air exhaust HEPA filter 221 can function normally.

In the present exemplary embodiment, the sterilant evacuation processing is partially different from the sterilant evacuation processing described in the first exemplary embodiment referring to FIG. 9, and different portions will thus be described below with reference to FIG. 9.

Since step S900 and step S904 in FIG. 9 are similar to those described in the first exemplary embodiment, description is omitted here.

In step S901, by the liquid feeding rotary pump 223, the sterilization apparatus 100 suctions all the liquid sterilant in the cartridge 205 by the pump, and feeds all the sterilant fed through the duct between the liquid sensor 204 and the liquid feeding rotary pump 223 to the air exhaust evaporation furnace 224 through the duct between the liquid feeding rotary pump 223 and the air exhaust evaporation furnace 224.

By the air exhaust evaporation furnace 224, the sterilization apparatus 100 heats by the heater installed in the air exhaust evaporation furnace 224 all the liquid sterilant (sterilant accumulated in the air exhaust evaporation furnace 224) to be fed through the duct between the liquid feeding rotary pump 223 and the air exhaust evaporation furnace 224, and gasifies all the sterilant. Subsequently, the gasified sterilant is fed to the sterilant degradation device 228 through the duct between the sterilant degradation device 228 and the air exhaust evaporation furnace 224.

Here, all the sterilant is gasified by the heater installed in the air exhaust evaporation furnace 224.

In step S902, the sterilant degradation device 228 degrades molecules of the sterilant contained in the gas fed through the duct between the sterilant degradation device 228 and the air exhaust evaporation furnace 224, and feeds the molecules generated by degradation to the air exhaust HEPA filter 221.

By the air exhaust HEPA filter 221, the sterilization apparatus 100 cleans the gasified sterilant fed through the duct between the sterilant degradation device 228 and the air exhaust HEPA filter 221, and the cleaned gas (containing the sterilant) passes through the duct between the sterilant degradation device 222 and the air exhaust HEPA filter 221 and is fed to the sterilant degradation device 222.

In step S903, the sterilant degradation device 222 degrades molecules of the sterilant contained in the gas fed through the duct between the sterilant degradation device 222 and the air exhaust HEPA filter 221, and discharges the molecules generated by degradation out of the sterilization apparatus 100.

In the first exemplary embodiment, in a case where the user turns off the main power, for example, during evaporation and degradation processing of the excessive hydrogen peroxide solution in the cartridge 205 set in the sterilization apparatus 100, operations of the air feeding pressure pump 209 stop. Thus, the hydrogen peroxide steam gasified in the air exhaust evaporation furnace 224 will accumulate in the duct between the air exhaust evaporation furnace 224 and the air exhaust HEPA filter 221, in the air exhaust HEPA filter 221, and in the duct between the air exhaust HEPA filter 221 and the sterilant degradation device 222 since air flow stops.

Thereafter, the hydrogen peroxide steam accumulating in the duct between the air exhaust evaporation furnace 224 and the air exhaust HEPA filter 221, in the air exhaust HEPA filter 221, and in the duct between the air exhaust HEPA filter 221 and the sterilant degradation device 222 may be cooled by the ambient temperature and may form dew condensation.

Thus, the air exhaust HEPA filter 221 may become extremely poor in air permeability and be broken easily when the air exhaust HEPA filter 221 absorbs liquid. In a case where the air feeding vacuum pump 220 and the air feeding pressure pump 209 are operated in this state, the air exhaust HEPA filter 221 may be clogged and may be broken as the air exhaust HEPA filter 221 cannot withstand the pressure.

The present exemplary embodiment can solve such a problem in the first exemplary embodiment.

That is, by providing the sterilant degradation device 228 between the air exhaust HEPA filter 221 and the air exhaust evaporation furnace 224, and degrading in the sterilant degradation device 228 the sterilant fed from the concentration furnace 208 to the air exhaust evaporation furnace 224 and the excessive sterilant in the cartridge 205 suctioned and fed to the air exhaust evaporation furnace 224, the sterilant is not liquefied easily in the air exhaust HEPA filter 221, and a product lifetime of the air exhaust HEPA filter 221 and a product lifetime of the sterilization apparatus 100 can be extended.

In the descriptions of the first exemplary embodiment and the second exemplary embodiment, in step S7021 in FIG. 7, the extraction needle 203-A is moved to the bottom or the proximity of the bottom in the cartridge 205, in step S704 or step S729 in FIG. 7, the liquid sterilant in the cartridge 205 is extracted, and in step S8032 in FIG. 8, the extraction needle 203-A is moved to the position in the cartridge 205 in which the extraction needle 203-A does not dip in the sterilant and in which the unsealing portion of the cartridge 205 is sealed (FIG. 15). However, it is possible to perform control so that, when the liquid sterilant in the cartridge 205 is extracted in step S704 or step S729 in FIG. 7, the extraction needle 203-A may be moved to the bottom or the proximity of the bottom in the cartridge 205, the liquid sterilant in the cartridge 205 may be extracted, and immediately after the extraction, the extraction needle 203-A may be moved to the position in the cartridge 205 in which the extraction needle 203-A does not dip in the sterilant and in which the unsealing portion of the cartridge 205 is sealed (FIG. 15).

As described above, according to the present exemplary embodiment, in a mechanism for inserting the extraction tube in the cartridge in which as much sterilant as doses for several sterilization processing operations is filled in a bottle, extracting as much sterilant as a dose from the cartridge, and performing the sterilization processing by using the extracted sterilant, it is possible to delay degradation of the sterilant in the cartridge in which the extraction tube is inserted and to prevent easy shortening of the period in which the sterilization processing that can exert a sufficient sterilization effect can be performed.

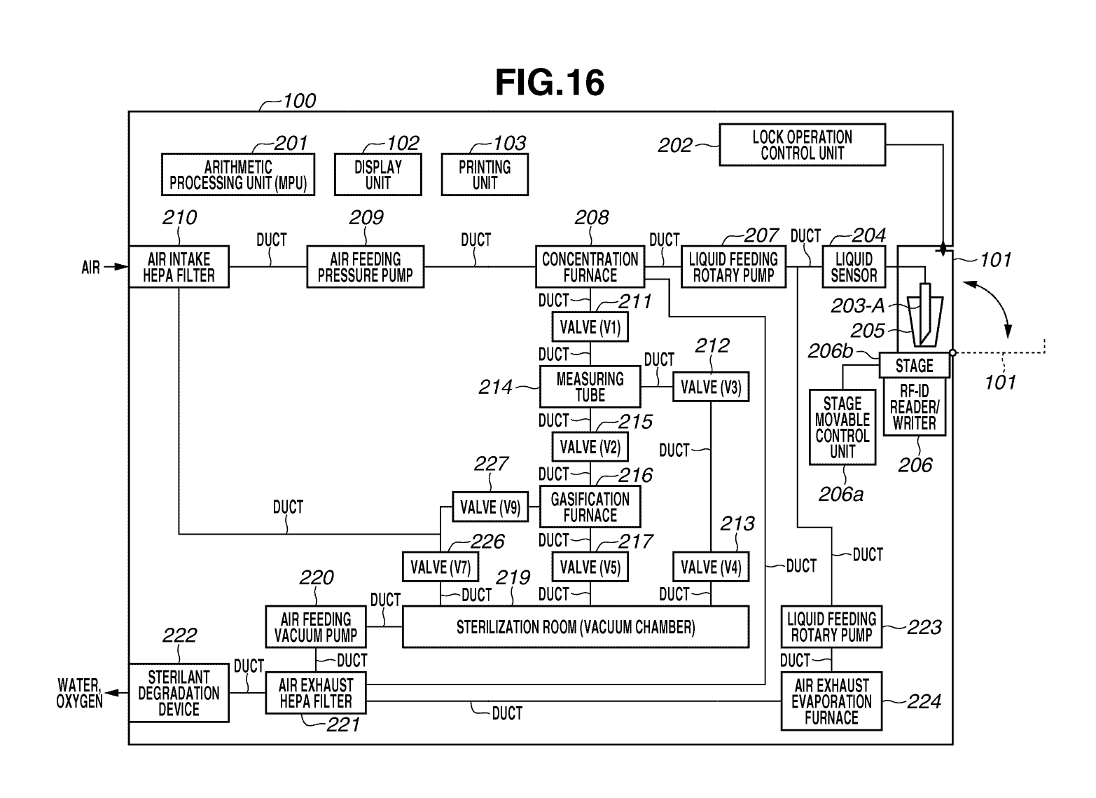

A sterilization apparatus according to a third exemplary embodiment will be described below with reference to FIG. 16.

As for the third exemplary embodiment, different portions from those in the sterilization apparatus described according to the first exemplary embodiment are described.

FIG. 16 illustrates an example of a hardware configuration of the sterilization apparatus according to the present exemplary embodiment.

A stage 206b and a stage operation control unit 206a are added to the sterilization apparatus 100 described according to the first exemplary embodiment, and the extraction needle operation control unit 203 is excluded from the sterilization apparatus 100 described according to the first exemplary embodiment.

The stage 206b is integrated with the RF-ID reader/writer 206, and the cartridge 205 is put on the stage 206b for use. The stage 206b can be moved up and down by the stage operation control unit 206a.

In the first exemplary embodiment, the extraction needle 203-A is inserted in the cartridge 205 under control of the extraction needle operation control unit 203. However, in the third exemplary embodiment, the stage 206b is moved upward to cause the fixed extraction needle 203-A to be inserted in the cartridge 205 under control of the stage operation control unit 206a, instead of control of the extraction needle operation control unit 203.

The sterilization apparatus 100 according to the third exemplary embodiment is similar to the sterilization apparatus 100 according to the first exemplary embodiment other than the above-described configuration.

That is, since the sterilization apparatus 100 according to the third exemplary embodiment is configured as illustrated in FIG. 16, the sterilization apparatus 100 according to the third exemplary embodiment is controlled in the following manner.

In a case where the screen illustrated in FIG. 11 is displayed on the display unit 102, and the user presses the button 1102 and opens the cartridge attachment door 101, the sterilization apparatus 100 performs processing for moving the stage 206b in a downward direction and thereafter releasing locking of the cartridge attachment door 101. Through this operation, the user can set the cartridge 205 in the cartridge attachment door 101.

In the first exemplary embodiment, the extraction needle 203-A is lowered and is inserted in the cartridge 205. However, in the third exemplary embodiment, in a corresponding step, the stage 206b is moved in an upward direction to cause the extraction needle 203-A to be inserted in the cartridge 205, instead of insertion of the extraction needle 203-A.

The moving amount of the stage 206b in the upward direction is equal to the moving amount of the extraction needle 203-A, which is moved downward to insert the extraction needle 203-A in the cartridge 205, described in the first exemplary embodiment.

Further, in the first exemplary embodiment, in the step for pulling the extraction needle 203-A out of the cartridge 205 and in the step for raising the extraction needle 203-A in the upper direction from the cartridge 205 and moving the extraction needle 203-A to the predetermined position in the cartridge 205 in which the extraction needle 203-A does not dip in the sterilant, the extraction needle 203-A is raised in the third exemplary embodiment.

In the third exemplary embodiment, the stage 206b is moved in the downward direction, instead of raising the extraction needle 203-A. The moving amount of the stage 206b is equal to the moving amount of the extraction needle 203-A in the first exemplary embodiment.

In this manner, in the third exemplary embodiment, the movement of the extraction needle 203-A in the first exemplary embodiment can be replaced with the movement of the stage 206b.

More specifically, the sterilization apparatus 100 extracting the sterilant from the cartridge containing the sterilant and sterilizing a sterilization target includes the extraction tube (extraction needle 203-A) extracting the sterilant from the cartridge, and the movement unit (206a) moving the cartridge so that a position of the extraction tube against the cartridge in a case of extracting the sterilant in the cartridge by the extraction tube and a position of the extraction tube against the cartridge in a case of waiting for extraction of the sterilant from the cartridge by the extraction tube to perform the following sterilization processing with the same cartridge may differ from each other.

This movement unit moves the cartridge to the position for extracting the sterilant in the cartridge by the extraction tube in a case of extracting the sterilant in the cartridge by the extraction tube, and moves the cartridge in an opposite direction of the moving direction of the cartridge moving for extraction of the sterilant in the cartridge in a case of waiting for extraction of the sterilant from the cartridge by the extraction tube to perform the following sterilization processing with the same cartridge.

Also, this movement unit moves the cartridge to cause the extraction tube to be inserted in the cartridge in a case of extracting the sterilant in the cartridge by the extraction tube, and moves the cartridge so that the extraction tube may be located at a position in which the extraction tube is not pulled out of the cartridge and in which the extraction tube does not dip in the sterilant in the cartridge on condition that the sterilant in the cartridge has been extracted by the inserted extraction tube.

Further, the movement unit moves the cartridge to the position for waiting for extraction of the sterilant from the cartridge by the extraction tube to perform the following sterilization processing with the same cartridge on condition that it is determined in step S8031 that the cartridge contains the predetermined amount of the sterilant required for the sterilization processing (YES in step S8031).

Further, the movement unit moves the cartridge to pull the extraction tube out of the cartridge on condition that it is determined in step S8031 that the cartridge does not contain the predetermined amount of the sterilant required for the sterilization processing (NO in step S8031).

The sterilization apparatus 100 further includes the disposal unit (the sterilant degradation device 222 or FIG. 9) disposing of the sterilant extracted from the cartridge. The movement unit moves the cartridge to pull the extraction tube out of the cartridge on condition that the sterilant in the cartridge has been extracted by the extraction tube to dispose of the sterilant contained in the cartridge by the disposal unit.

As described above, according to the present exemplary embodiment, in a mechanism for inserting the extraction tube in the cartridge in which as much sterilant as doses for several sterilization processing operations is filled in a bottle by moving up and down the stage on which the cartridge is put, extracting as much sterilant as a dose from the cartridge, and performing the sterilization processing by using the extracted sterilant, it is possible to delay degradation of the sterilant in the cartridge in which the extraction tube is inserted and to prevent easy shortening of the period in which the sterilization processing that can exert a sufficient sterilization effect can be performed.

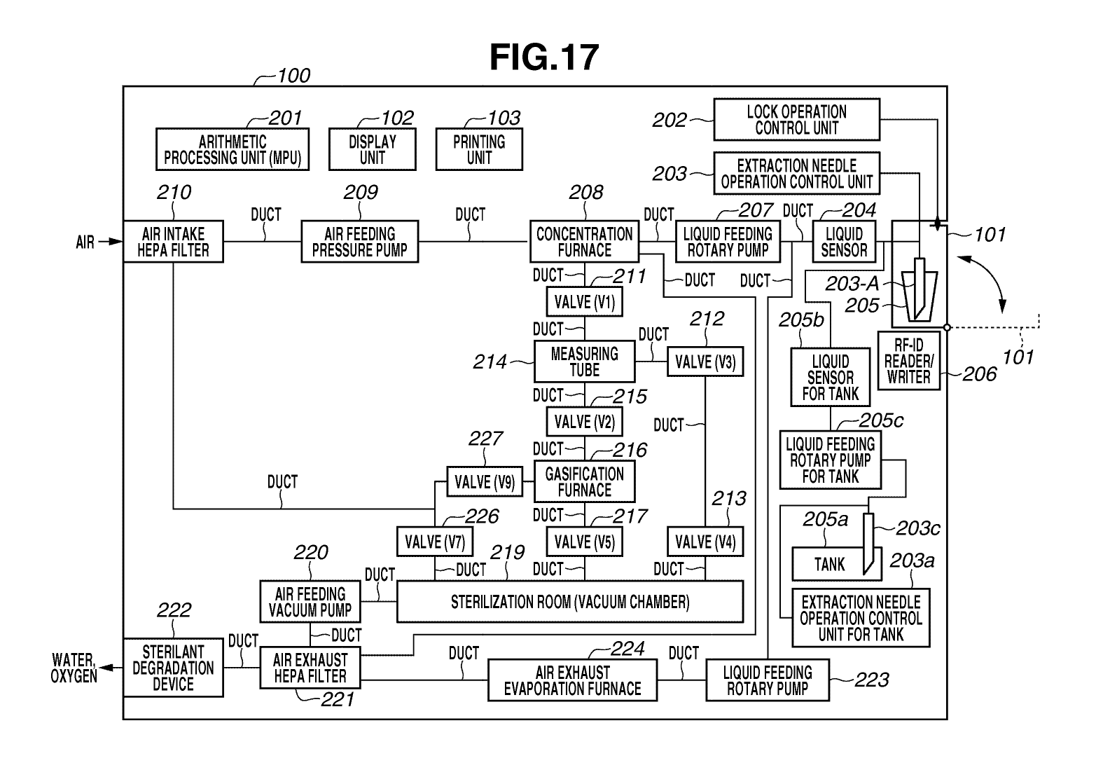

A sterilization apparatus according to a fourth exemplary embodiment will be described below with reference to FIG. 17.

As for the fourth exemplary embodiment, different portions from those in the sterilization apparatus described according to the first exemplary embodiment are described.

FIG. 17 illustrates an example of a hardware configuration of the sterilization apparatus according to the fourth exemplary embodiment.

To the sterilization apparatus 100 described according to the first exemplary embodiment are added a tank 205a, an extraction needle operation control unit for a tank 203a, an extraction needle for a tank 203c, a liquid sensor for a tank 205b, and a liquid feeding rotary pump for a tank 205c.

The extraction needle for a tank 203c is moved to be inserted in the tank 205a by the extraction needle operation control unit for a tank 203a, and the sterilant in the cartridge 205 is extracted from the cartridge 205 via the extraction tube (extraction needle 203-A) by operating the liquid feeding rotary pump for a tank 205c.

When it is confirmed by the liquid sensor for a tank 205b that the sterilant has been extracted from the cartridge 205, the sterilant suctioned from the cartridge 205 is accumulated in the tank 205a.

In other words, although sealing of the cartridge 205 is maintained by movement of the extraction needle 203-A by the extraction needle operation control unit 203 in the first exemplary embodiment, sealing of the tank 205a is maintained by movement of the extraction needle for a tank 203c by the extraction needle operation control unit for a tank 203a in the fourth exemplary embodiment.

The sterilization apparatus 100 according to the fourth exemplary embodiment is similar to the sterilization apparatus 100 according to the first exemplary embodiment other than the aforementioned configuration.

In other words, since the sterilization apparatus 100 according to the fourth exemplary embodiment is configured as illustrated in FIG. 17, the sterilization apparatus 100 according to the fourth exemplary embodiment is controlled in the following manner.

In the first exemplary embodiment, the sterilant is extracted from the cartridge 205 each time of the sterilization processing. However, in the fourth exemplary embodiment, all the sterilant in the cartridge 205 is extracted and accumulated in the tank 205a before executing the sterilization processing (beforehand).

In the step for lowering the extraction needle 203-A in the first exemplary embodiment, the extraction needle for the tank 203c is lowered in the fourth exemplary embodiment. In addition, the moving amount of the extraction needle for a tank 203c to be lowered is equal to the moving amount of the extraction needle 203-A to be lowered in the first exemplary embodiment.

Further, in the step for raising the extraction needle 203-A in the first exemplary embodiment, the extraction needle for a tank 203c is raised in the fourth exemplary embodiment. In addition, the moving amount of the extraction needle for a tank 203c to be raised is equal to the moving amount of the extraction needle 203-A to be raised in the first exemplary embodiment.

In the first exemplary embodiment, the extraction needle 203-A is lowered or raised with respect to the cartridge 205 to seal the cartridge 205. However, in the fourth exemplary embodiment, the extraction needle for a tank 203c is lowered or raised with respect to the tank 205a to seal the tank 205a.

Thus, in the fourth exemplary embodiment, movement of the extraction needle 203-A in the first exemplary embodiment is replaced with movement of the extraction needle for a tank 203c, and the cartridge 205 as a target for extraction of the sterilant in the first exemplary embodiment is replaced with the tank 205a as a target for extraction of the sterilant.

As described above, according to the present exemplary embodiment, in a mechanism for inserting the extraction needle for a tank 203c (extraction tube) in the sterilant in the tank accumulating the sterilant extracted from the cartridge under control of the extraction needle operation control unit for a tank, extracting as much sterilant as a dose from the tank, and performing the sterilization processing by using the extracted sterilant, it is possible to delay degradation of the sterilant in the tank in which the extraction tube is inserted and to prevent easy shortening of the period in which the sterilization processing that can exert a sufficient sterilization effect can be performed.

A sterilization apparatus according to a fifth exemplary embodiment will be described below with reference to FIG. 17.

As for the fifth exemplary embodiment, different portions from those in the sterilization apparatus described in the fourth exemplary embodiment are described.

In the fourth exemplary embodiment, the extraction needle for a tank 203c is inserted in the tank 205a under control of the extraction needle operation control unit for a tank 203a. However, in the fifth exemplary embodiment, a stage provided at a lower portion of the tank 205a to support the tank 205a is moved upward to cause the fixed extraction needle for a tank 203c to be inserted in the tank 205a under control of a stage operation control unit moving the stage up and down, instead of control of the extraction needle operation control unit for a tank 203a.

In other words, in the third exemplary embodiment, the fixed extraction needle 203-A is caused to be inserted in the cartridge 205 by moving up and down the stage 206b at the lower portion of the cartridge 205. However, in the fifth exemplary embodiment, the fixed extraction needle for a tank 203c is caused to be inserted in the tank 205a by moving up and down the stage at the lower portion of the tank 205a.

The sterilization apparatus 100 according to the fifth exemplary embodiment is similar to the sterilization apparatus 100 according to the first exemplary embodiment other than the configuration described above.

In this manner, in the third exemplary embodiment, the movement of the extraction needle 203-A in the first exemplary embodiment is replaced with the movement of the stage 206b.

In this manner, in the fifth exemplary embodiment, the movement of the stage 206b on which the cartridge 205 is to be put in the third exemplary embodiment is replaced with the movement of the stage on which the tank 205a is to be put. Further, the up-down moving amount of this stage is equal to that in the third exemplary embodiment.

As described above, according to the present exemplary embodiment, in a mechanism for inserting the extraction tube in the tank in which as much sterilant as doses for several sterilization processing operations is filled in a tank by moving up and down the stage on which the tank is put, extracting as much sterilant as a dose from the tank, and performing the sterilization processing by using the extracted sterilant, it is possible to delay degradation of the sterilant in the tank in which the extraction tube is inserted and to prevent easy shortening of the period in which the sterilization processing that can exert a sufficient sterilization effect can be performed.

More specifically, as described above in the fourth and fifth exemplary embodiments, the sterilization apparatus 100 extracting the sterilant from the tank 205a accumulating the sterilant and sterilizing a sterilization target includes the extraction tube 203c extracting the sterilant from the tank and the movement unit (203a or the stage supporting the tank) moving the extraction tube or the tank so that a position of the extraction tube with respect to the tank in a case of extracting the sterilant in the tank by the extraction tube and a position of the extraction tube with respect to the tank in a case of waiting for extraction of the sterilant from the tank by the extraction tube to perform the following sterilization processing with the same tank may differ from each other.

The movement unit moves the extraction tube or the tank to the position for extracting the sterilant in the cartridge by the extraction tube in a case of extracting the sterilant in the cartridge by the extraction tube, and moves the extraction tube or the tank in an opposite direction of the moving direction of the extraction tube or the tank moving for extraction of the sterilant in the cartridge in a case of waiting for extraction of the sterilant from the cartridge by the extraction tube to perform the following sterilization processing with the same cartridge.

Further, this movement unit moves the extraction tube or the tank to cause the extraction tube to be inserted in the cartridge in a case of extracting the sterilant in the cartridge by the extraction tube, and moves the extraction tube or the tank so that the extraction tube may be located at a position in which the extraction tube is not pulled out of the cartridge and in which the extraction tube does not dip in the sterilant in the cartridge on condition that the sterilant in the cartridge has been extracted by the inserted extraction tube.

The sterilization apparatus 100 further includes a determination method (S8031) determining whether the cartridge after extraction of the sterilant by the extraction tube contains the predetermined amount of the sterilant required for the sterilization processing based on the result of extraction of the sterilant from the cartridge by the extraction tube. The movement unit moves the extraction tube or the tank to the position for waiting for extraction of the sterilant from the cartridge by the extraction tube to perform the following sterilization processing with the same cartridge on condition that it is determined by the determination method that the cartridge contains the predetermined amount of the sterilant required for the sterilization processing.

Further, the movement unit moves the extraction tube or the tank to pull the extraction tube out of the cartridge on condition that it is determined by the determination method that the cartridge does not contain the predetermined amount of the sterilant required for the sterilization processing.

The sterilization apparatus 100 further includes the disposal unit (the sterilant degradation device 222 or FIG. 9) disposing of the sterilant extracted from the cartridge. The movement unit moves the extraction tube or the tank to pull the extraction tube out of the cartridge on condition that the sterilant in the cartridge has been extracted by the extraction tube to dispose of the sterilant contained in the cartridge by the disposal unit.

According to the above-described exemplary embodiments, it is possible to delay degradation of the sterilant in the cartridge or the tank in which the extraction tube is inserted.

While the above-described exemplary embodiments have been provided, it is to be understood that these embodiments are not seen to be limiting. The scope of the following claims is to be accorded the broadest interpretation so as to encompass all such modifications and equivalent structures and functions.

This application claims the benefit of Japanese Patent Application No. 2013-106660 filed May 20, 2013, and No. 2012-272252 filed Dec. 13, 2012, which are hereby incorporated by reference herein in their entirety.

What is claimed is:

1. A sterilization apparatus extracting sterilant from a cartridge containing the sterilant and sterilizing a sterilization target, the sterilization apparatus comprising:
an extraction tube configured to extract the sterilant from the cartridge; and
a movement unit configured to move the extraction tube so that a position of the extraction tube with respect to the cartridge in a case of extracting the sterilant in the cartridge by the extraction tube and a position of the extraction tube with respect to the cartridge in a case of waiting for extraction of the sterilant from the cartridge by the extraction tube to perform following sterilization processing with the same cartridge differ from each other.

2. The sterilization apparatus according to claim 1, wherein the movement unit moves the extraction tube to a position for extracting the sterilant in the cartridge by the extraction tube in a case of extracting the sterilant in the cartridge by the extraction tube, and moves the extraction tube in an opposite direction of a moving direction of the extraction tube moving for extraction of the sterilant in the cartridge in a case of waiting for extraction of the sterilant from the cartridge by the extraction tube to perform following sterilization processing with the same cartridge.

3. The sterilization apparatus according to claim 1, wherein the movement unit moves the extraction tube to cause the extraction tube to be inserted in the cartridge in a case of extracting the sterilant in the cartridge by the extraction tube, and moves the extraction tube so that the extraction tube is located at a position in which the extraction tube is not pulled out of the cartridge and in which the extraction tube does not dip in the sterilant in the cartridge if the sterilant in the cartridge has been extracted by the inserted extraction tube.

4. The sterilization apparatus according to claim 1, further comprising:
a determination unit configured to determine whether the cartridge, after extraction of the sterilant by the extraction tube, contains a predetermined amount of the sterilant required for sterilization processing based on a result of extraction of the sterilant from the cartridge by the extraction tube,
wherein the movement unit moves the extraction tube to a position for waiting for extraction of the sterilant from the cartridge by the extraction tube to perform following sterilization processing with the same cartridge if it the determination unit determines that the cartridge contains the predetermined amount of the sterilant required for the sterilization processing.

5. The sterilization apparatus according to claim 4, wherein the movement unit moves the extraction tube to pull the extraction tube out of the cartridge if the determination unit determines that the cartridge does not contain the predetermined amount of the sterilant required for the sterilization processing.

6. The sterilization apparatus according to claim 1, further comprising:
a disposal unit configured to dispose of the sterilant extracted from the cartridge,
wherein the movement unit moves the extraction tube to pull the extraction tube out of the cartridge if the sterilant in the cartridge has been extracted by the extraction tube to dispose of the sterilant contained in the cartridge.

7. A sterilization apparatus extracting sterilant from a cartridge containing the sterilant and sterilizing a sterilization target, the sterilization apparatus comprising:
an extraction tube configured to extract the sterilant from the cartridge; and
a movement unit configured to move the cartridge so that a position of the extraction tube with respect to the cartridge in a case of extracting the sterilant in the cartridge by the extraction tube and a position of the extraction tube with respect to the cartridge in a case of waiting for extraction of the sterilant from the cartridge by the extraction tube to perform following sterilization processing with the same cartridge differ from each other.

8. The sterilization apparatus according to claim 7, wherein the movement unit moves the cartridge to a position for extracting the sterilant in the cartridge by the extraction tube in a case of extracting the sterilant in the cartridge by the extraction tube, and moves the cartridge in an opposite direction of a moving direction of the cartridge moving for extraction of the sterilant in the cartridge in a case of waiting for extraction of the sterilant from the cartridge by the extraction tube to perform following sterilization processing with the same cartridge.

9. The sterilization apparatus according to claim 7, wherein the movement unit moves the cartridge to cause the extraction tube to be inserted in the cartridge in a case of extracting the sterilant in the cartridge by the extraction tube, and moves the cartridge so that the extraction tube is located at a position in which the extraction tube is not pulled out of the cartridge and in which the extraction tube does not dip in the sterilant in the cartridge on condition that the sterilant in the cartridge has been extracted by the inserted extraction tube.

10. The sterilization apparatus according to claim 7, further comprising:
a determination unit configured to determine whether the cartridge, after extraction of the sterilant by the extraction tube, contains a predetermined amount of the sterilant required for sterilization processing based on a result of extraction of the sterilant from the cartridge by the extraction tube,
wherein the movement unit moves the cartridge to a position for waiting for extraction of the sterilant from the cartridge by the extraction tube to perform following sterilization processing with the same cartridge the determination unit determines that the cartridge contains the predetermined amount of the sterilant required for the sterilization processing.

11. The sterilization apparatus according to claim 10, wherein the movement unit moves the cartridge to pull the extraction tube out of the cartridge if the determination unit determines that the cartridge does not contain the predetermined amount of the sterilant required for the sterilization processing.

12. The sterilization apparatus according to claim 7, further comprising:
a disposal unit configured to dispose of the sterilant extracted from the cartridge,
wherein the movement unit moves the cartridge to pull the extraction tube out of the cartridge if the sterilant in the cartridge has been extracted by the extraction tube to dispose of the sterilant contained in the cartridge.

13. A sterilization apparatus extracting sterilant from a tank accumulating the sterilant and sterilizing a sterilization target, the sterilization apparatus comprising:
an extraction tube configured to extract the sterilant from the tank; and
a movement unit configured to move the extraction tube or the tank so that a position of the extraction tube with respect to the tank in a case of extracting the sterilant in the tank by the extraction tube and a position of the extraction tube with respect to the tank in a case of waiting for extraction of the sterilant from the tank by the extraction tube to perform following sterilization processing with the same tank differ from each other.

14. The sterilization apparatus according to claim 13, wherein the movement unit moves the extraction tube or the tank to a position for extracting the sterilant in the tank by the extraction tube in a case of extracting the sterilant in the tank by the extraction tube, and moves the extraction tube or the tank in an opposite direction of a moving direction of the extraction tube or the tank moving for extraction of the sterilant in the tank in a case of waiting for extraction of the sterilant from the tank by the extraction tube to perform following sterilization processing with the same tank.

15. The sterilization apparatus according to claim 13, wherein the movement unit moves the extraction tube or the tank to cause the extraction tube to be inserted in the tank in a case of extracting the sterilant in the tank by the extraction tube, and moves the extraction tube or the tank so that the extraction tube is located at a position in which the extraction tube is not pulled out of the tank and in which the extraction tube does not dip in the sterilant in the tank on condition that the sterilant in the tank has been extracted by the inserted extraction tube.

16. The sterilization apparatus according to claim 13, further comprising:
a determination unit configured to determine whether the tank, after extraction of the sterilant by the extraction tube, contains a predetermined amount of the sterilant required for sterilization processing based on a result of extraction of the sterilant from the tank by the extraction tube,
wherein the movement unit moves the extraction tube or the tank to a position for waiting for extraction of the sterilant from the tank by the extraction tube to perform following sterilization processing with the same tank if the determination unit determines that the tank contains the predetermined amount of the sterilant required for the sterilization processing.

17. The sterilization apparatus according to claim 16, wherein the movement unit moves the extraction tube or the tank to pull the extraction tube out of the tank if the determination unit determines that the tank does not contain the predetermined amount of the sterilant required for the sterilization processing.

18. The sterilization apparatus according to claim 13, further comprising:
a disposal unit configured to dispose of the sterilant extracted from the tank,
wherein the movement unit moves the extraction tube or the tank to pull the extraction tube out of the tank if the sterilant in the tank has been extracted by the extraction tube to dispose of the sterilant contained in the tank.

19. A sterilization method in a sterilization apparatus provided with an extraction tube for extracting a sterilant, the sterilization method comprising:
extracting the sterilant from a cartridge; and moving the extraction tube so that a position of the extraction tube with respect to the cartridge in a case of extracting the sterilant in the cartridge by the extraction tube and a position of the extraction tube with respect to the cartridge in a case of waiting for extraction of the sterilant from the cartridge by the extraction tube to perform following sterilization processing with the same cartridge differ from each other.

20. A sterilization method in a sterilization apparatus provided with an extraction tube for extracting a sterilant, the sterilization method comprising:
extracting the sterilant from a cartridge; and
moving the cartridge so that a position of the extraction tube with respect to the cartridge in a case of extracting the sterilant in the cartridge by the extraction tube and a position of the extraction tube with respect to the cartridge in a case of waiting for extraction of the sterilant from the cartridge by the extraction tube to perform following sterilization processing with the same cartridge differ from each other.

21. A sterilization method in a sterilization apparatus provided with an extraction tube for extracting a sterilant, the sterilization method comprising:
extracting the sterilant from a tank; and
moving the extraction tube or the tank so that a position of the extraction tube with respect to the tank in a case of extracting the sterilant in the tank by the extraction tube and a position of the extraction tube with respect to the tank in a case of waiting for extraction of the sterilant from the tank by the extraction tube to perform following sterilization processing with the same tank differ from each other.
